(19) United States
(12) Patent Application Publication
(10) Pub. No.: US 2026/0203798 A1
(43) Pub. Date: Jul. 16, 2026

(54) SYSTEM AND METHOD FOR AUTOMATIC TAGGING OF IMAGES AND VIDEO IN AN OPERATIVE REPORT

(71) Applicant: VAIM Technologies LLC, Oceanport, NJ (US)

(72) Inventors: Manjeet DHARIWAL, Victor, NY (US); Aaron FEILER, Fair Haven, NJ (US); Inderpal Singh SARKARIA, Dallas, TX (US)

(21) Appl. No.: 19/564,915

(22) Filed: Mar. 12, 2026

Related U.S. Application Data
(63) Continuation-in-part of application No. 18/133,293, filed on Apr. 11, 2023, now Pat. No. 12,579,774.
(60) Provisional application No. 63/329,921, filed on Apr. 12, 2022.

(51) Int. Cl. G06Q 30/04; G16H 30/40; G16H 70/20

(52) U.S. Cl. CPC G06Q 30/04; G16H 30/40; G16H 70/20

(57) ABSTRACT

Systems and methods for automatic tagging of images and video in surgical streams are described. A plurality of machine learning models, trained on annotated surgical data, are used to extract salient images and video clips from surgical video streams. In addition, speech transcription models process audio streams to generate transcriptions that are then associated with the tagged media. Subsequently, the system synchronizes the multimodal data and generates structured operative records. After synchronization, billing rules are applied to produce accurate billing reports. Applications of the system include improving surgical documentation, reducing administrative burden, enhancing billing accuracy, and accelerating revenue cycles in healthcare environments.

### CROSS-REFERENCE TO RELATED APPLICATIONS

This application is a continuation-in-part of U.S. patent application Ser. No. 18/133,293, filed Apr. 11, 2023, entitled “SYSTEM AND METHOD FOR AUTOMATIC TAGGING OF IMAGES AND VIDEO IN AN OPERATIVE REPORT,” which claims priority to U.S. Provisional Patent Application No. 63/329,921, filed Apr. 12, 2022, entitled “SYSTEM AND METHOD FOR AUTOMATIC TAGGING OF IMAGES AND VIDEO IN AN OPERATIVE REPORT,” the entire disclosures of which are incorporated herein in their entirety by reference.

### TECHNICAL FIELD

Aspects of this document relate generally to systems and methods for automatic tagging of images and video in a surgical stream for keyboard-less generation and summarization of surgical operative report and automated generation of billing codes for surgical procedures, and more particularly to a combination of AI-driven natural language and image processing systems for dictation, voice recognition, and image and video capture for synchronously generating and assessing operative records and medical billing codes during surgical procedures.

### BACKGROUND

An operative report is a report written in a patient's medical record to document the details of a surgery. Conventionally, the operative report is dictated right after a surgical procedure and later transcribed into the patient's record. The information in the operative report includes preoperative and postoperative diagnosis and the condition of the patient after the surgery. It is necessary for other healthcare professionals immediately attending the postoperative recovery of the patient.

The operative report is produced by a surgeon or other physician(s) who have participated in the surgery and contains a detailed account of the findings, the procedure used, the specimens removed, and the preoperative and postoperative diagnoses. The operative report may also include identifying information of the patient and names of the primary performing surgeon and any assistants. In the medicolegal context, the operative report serves to document the steps that were and were not taken to complete the surgery without unintended injury. As lawsuits and trials occur years after surgery when memories have faded, a well written operative report detailing the steps taken to avoid surgical complication is crucial to a successful defense, as it allows the defendant to reconstruct a surgery performed years earlier. It is very important that the operative report describes the steps taken in chronological order.

Surgeons typically document operative events using dictation services. Dictated reports are frequently incomplete or delayed. Certain elements must be included to ensure that the operative report satisfies institutional and national standards. After transcribing, the surgeon must review, correct errors, and ultimately “verify” the dictated report. There may be delays to the time of initial dictation by the surgeon, transcription of the dictated report, or final verification by the surgeon. Delays or lack of structure in dictation may also increase the likelihood that important elements may be missing from the final report.

Tagged multimedia—images and video clips of the surgery—form a very important part of the operative report. They provide critical evidence required to reconstruct what happened during a surgical procedure. Conventionally, a surgeon has to review the entire surgical video, which could be hours long, after surgery and manually tag or insert salient images or video clips into the operative report. Not only is this process time consuming, it is error-prone and puts the onus for accuracy and completeness on the surgeon.

Beyond operative report generation, the medical billing process for surgical procedures presents additional significant challenges. Conventionally, after a surgical procedure is completed and the operative report is generated, an administrative assistant, medical coder, or billing specialist must review the surgical video recordings, the operative report, and other medical documentation to determine appropriate billing codes for the procedure. This conventional billing code generation process is highly labor-intensive, time-consuming, and prone to human error.

Medical billing codes, including Current Procedural Terminology (CPT) codes maintained by the American Medical Association, Healthcare Common Procedure Coding System (HCPCS) codes, and International Classification of Diseases (ICD) codes, must accurately reflect the specific surgical procedures performed, the complexity of the surgery, the time spent, the equipment used, and other billable events that occurred during the procedure. Inaccurate coding can result in claim denials, delayed reimbursements, compliance violations, and potential fraud allegations. The complexity of surgical billing is compounded by the frequent updates to billing code databases, with codes being added, modified, or deprecated on a regular basis.

In conventional approaches, the administrative personnel reviewing surgical videos and reports may lack the medical expertise to fully understand the nuances of surgical procedures, leading to miscoding. Additionally, the time delay between the surgical procedure and the billing code generation can result in lost revenue due to missed billing opportunities or inaccurate recollection of procedural details. The manual review process also creates bottlenecks in the revenue cycle, delaying reimbursements to healthcare providers.

Accordingly, a need in the art exists for improved automatic generation of operative reports including automatically extracting and tagging salient images or video clips of the surgical procedure. A further need exists in the art for automated systems and methods that can generate accurate billing codes in real-time or near real-time during or immediately after surgical procedures, based on AI-driven analysis of surgical video streams and surgeon narration, thereby improving billing accuracy, reducing administrative burden, accelerating the revenue cycle, and ensuring compliance with billing regulations.

### SUMMARY

At least the above-discussed need is addressed, and technical solutions are achieved in the art by various embodiments of the present invention. In some embodiments, a system for generating a billing report for a surgical procedure comprises one or more computer accessible storage devices configured to store instructions; and one or more processors communicatively connected to the one or more computer accessible storage devices and configured to execute the instructions to receive at least one of a surgical video stream or a surgical audio stream for the surgical procedure; generate, when the surgical audio stream is received, a transcription stream from the surgical audio stream using incremental decoding; extract a plurality of multimodal features from at least one of the surgical video stream, the surgical audio stream, or the transcription stream; input the plurality of multimodal features into a trained billing code machine learning model to output a set of predicted billing codes each having associated therewith at least one of (i) a timestamp or time range or (ii) a confidence score; determine, based on a case context derived from at least one of scheduling metadata, encounter metadata, the transcription stream, or the surgical video stream, one or more modifiers applicable to at least one predicted billing code; select a versioned rule set for at least a subset of the predicted billing codes, the versioned rule set defining one or more of (i) eligibility conditions, (ii) modifier effects, (iii) bundling or unbundling outcomes, or (iv) amount computation rules; transform the set of predicted billing codes into a plurality of billing lines by at least applying the versioned rule set and the one or more modifiers to the predicted billing codes; and generate the billing report including the plurality of billing lines.

In some embodiments, receiving the at least one of the surgical video stream or the surgical audio stream comprises receiving both the surgical video stream and the surgical audio stream, and generating the transcription stream comprises time-aligning the transcription stream to the surgical video stream.

In some embodiments, generating the transcription stream using incremental decoding comprises producing partial transcriptions at a bounded latency and revising at least one partial transcription in response to receipt of additional audio samples.

In some embodiments, extracting the plurality of multimodal features comprises extracting at least one video feature comprising at least one of instrument features, anatomical structure features, motion features, or action recognition features.

In some embodiments, extracting the plurality of multimodal features comprises extracting at least one audio-derived feature comprising at least one of a keyword feature, a medical entity feature, a semantic embedding feature, or a temporal alignment feature between a transcription excerpt and a time range.

In some embodiments, trained billing code machine learning model comprises a multimodal neural network configured to fuse a video feature representation and a transcription feature representation to output the set of predicted billing codes.

In some embodiments, determining the one or more modifiers comprises determining an emergent-versus-scheduled modifier in response to detecting at least one emergent workflow indicator in at least one of the scheduling metadata, the encounter metadata, the transcription stream, or the surgical video stream.

In some embodiments, determining the one or more modifiers comprises resolving conflicts among a plurality of candidate modifiers using a stored modifier precedence table associated with at least one of a payer identifier or an institution identifier.

In some embodiments, selecting the versioned rule set comprises selecting the versioned rule set based on at least one of an institution identifier, a payer identifier, a specialty identifier, or a site-of-service identifier.

In some embodiments, transforming the set of predicted billing codes into the plurality of billing lines comprises applying at least one eligibility condition of the versioned rule set to suppress at least one predicted billing code.

In some embodiments, transforming the set of predicted billing codes into the plurality of billing lines comprises applying at least one bundling or unbundling outcome of the versioned rule set to replace at least one predicted billing code with a different billing code or to add at least one additional billing code.

In some embodiments, the amount computation rules comprise participant allocation rules that allocate at least a portion of an amount among a plurality of participants of the surgical procedure.

In some embodiments, the plurality of participants include two or more of a primary surgeon, an assistant surgeon, a co-surgeon, a resident, a fellow, an anesthesiologist, a certified registered nurse anesthetist, a circulating nurse, a scrub nurse, or a technician, and wherein generating the billing report comprises generating a participant attribution record for at least one billing line.

In some embodiments, the one or more processors are further configured to determine participant roles using at least one of scheduled staffing data, electronic health record participation logs, badge-in and badge-out logs, intraoperative device usage logs, transcribed speech indicating role participation, or video-based recognition of role indicators.

In some embodiments, the versioned rule set is compiled into an evaluation graph configured for deterministic traversal order, and transforming the set of predicted billing codes into the plurality of billing lines comprises executing the evaluation graph using cached intermediate results reused across a plurality of billing lines to reduce end-to-end billing report generation latency.

In some embodiments, the billing report includes, for each billing line, at least one of a timestamp or time range, a confidence score, or an evidence link that references at least one of a video segment identifier, an audio segment identifier, or a transcription excerpt identifier.

In some embodiments, the one or more processors are further configured to store, in association with at least one billing line, a provenance record identifying at least one of a model version identifier for the trained billing code machine learning model, a feature extraction pipeline identifier, a version identifier of the versioned rule set, or a modifier determination logic version identifier.

In some embodiments, the one or more processors are further configured to generate, using a large language model, a justification text field for at least one billing line. In some embodiments, generating the justification text field comprises inserting at least one standardized phrase from a versioned phrase library and enforcing an evidence-anchoring constraint in which each sentence of the justification text field includes a reference to at least one evidence item.

In some embodiments, a processor-implemented method of generating a billing report for a surgical procedure comprises receiving at least one of a surgical video stream or a surgical audio stream for the surgical procedure; generating, when the surgical audio stream is received, a transcription stream from the surgical audio stream using incremental decoding; extracting a plurality of multimodal features from at least one of the surgical video stream, the surgical audio stream, or the transcription stream; inputting the plurality of multimodal features into a trained billing code machine learning model to output a set of predicted billing codes each having associated therewith at least one of (i) a timestamp or time range or (ii) a confidence score; determining, based on a case context derived from at least one of scheduling metadata, encounter metadata, the transcription stream, or the surgical video stream, one or more modifiers applicable to at least one predicted billing code; selecting a versioned rule set for at least a subset of the predicted billing codes, the versioned rule set defining one or more of (i) eligibility conditions, (ii) modifier effects, (iii) bundling or unbundling outcomes, or (iv) amount computation rules; transforming the set of predicted billing codes into a plurality of billing lines by at least applying the versioned rule set and the one or more modifiers to the predicted billing codes; and generating the billing report including the plurality of billing lines.

In some embodiments, a non-transitory computer-readable storage medium stores instructions that, when executed by one or more processors, cause the one or more processors to perform the method of generating a billing report for a surgical procedure.

Various embodiments of the present invention may include methods, systems, devices, or machines that are or include combinations or subsets of any one or more of the methods, systems, devices, or machines and associated features thereof summarized above or otherwise described herein.

### BRIEF DESCRIPTION OF THE DRAWINGS

Embodiments will be discussed hereafter using reference to the included drawings, briefly described below, wherein like designations refer to like elements:

FIG. 1 shows a computing device system, according to embodiments of the invention;

FIG. 2 shows another computing device system, according to embodiments of the invention;

FIG. 3 shows an operative record generation system, according to some embodiments of the present invention;

FIG. 4 shows an exemplar user interface screen for the operative record generation system, according to some embodiments of the present invention;

FIGS. 5A-5D shows an exemplar workflow for the operative record generation system, according to some embodiments of the present invention;

FIG. 6 shows an exemplar operative record generated by the operative record generation system, according to some embodiments of the present invention;

FIG. 7 shows an exemplar user interface for assessing and managing credentials based on operative records generated by the operative record generation system, according to some embodiments of the present invention;

FIG. 8 shows a flowchart of a method of generating an operative record, according to some embodiments of the present invention;

FIG. 9 shows a flowchart of another method of generating an operative record, according to some embodiments of the present invention;

FIG. 10 shows a flowchart of a method of generating a speech machine learning model, according to some embodiments of the present invention; and

FIG. 11 shows a flowchart of a method of generating a video and image machine learning model, according to some embodiments of the present invention.

FIG. 12 shows an automated billing report generation system integrated with the operative record generation system, according to some embodiments of the present invention;

FIG. 13 shows a flowchart of a method of generating a billing code machine learning model, according to some embodiments of the present invention;

FIG. 14 shows a flowchart of a method of automatically generating billing codes for a surgical procedure, according to some embodiments of the present invention;

FIG. 15 shows an exemplar billing code report generated by the automated billing report generation system, according to some embodiments of the present invention;

FIG. 16 shows a flowchart of a method of applying billing rules and determining modifiers for the generated billing codes for the surgical procedure, according to some embodiments of the present invention;

FIG. 17 shows a flowchart of a method of using an RVU optimization engine to analyze and optimize the generated billing codes for the surgical procedure, according to some embodiments of the present invention; and

FIG. 18 shows a flowchart of a method of using generating narratives and justifications for the generated billing codes for the surgical procedure, according to some embodiments of the present invention.

### DETAILED DESCRIPTION

Implementations/embodiments disclosed herein (including those not expressly discussed in detail) are not limited to the particular components or procedures described herein. Additional or alternative components, assembly procedures, and/or methods of use consistent with the intended systems & methods for synchronous automated generation of operative reports may be utilized in any implementation. This may include any materials, components, sub-components, methods, sub-methods, steps, and so forth.

As used herein, the term “input field” includes a “selector.” For example, a button or space on a user interface in which a user may move a cursor to and click to make a selection, and a checkbox field, and other similar fields, as well as alphanumeric input fields, are all “input fields” as used herein.

In some embodiments, an operative record generation system provides synchronous summarization of an operative procedure with video and image tagging. It should be noted that the invention is not limited to these or any other examples provided herein, which are referred to for purposes of illustration only.

In this regard, in the descriptions herein, certain specific details are set forth in order to provide a thorough understanding of various embodiments of the invention. However, one skilled in the art will understand that the invention may be practiced at a more general level without one or more of these details. In other instances, well-known structures have not been shown or described in detail to avoid unnecessarily obscuring descriptions of various embodiments of the invention.

Any reference throughout this specification to “one embodiment”, “an embodiment”, “an example embodiment”, “an illustrated embodiment”, “a particular embodiment”, and the like means that a particular feature, structure or characteristic described in connection with the embodiment is included in at least one embodiment. Thus, any appearance of the phrase “in one embodiment”, “in an embodiment”, “in an example embodiment”, “in this illustrated embodiment”, “in this particular embodiment”, or the like in this specification is not necessarily all referring to one embodiment or a same embodiment. Furthermore, the particular features, structures or characteristics of different embodiments may be combined in any suitable manner to form one or more other embodiments.

Unless otherwise explicitly noted or required by context, the word “or” is used in this disclosure in a non-exclusive sense. In addition, unless otherwise explicitly noted or required by context, the word “set” is intended to mean one or more. For example, the phrase, “a set of objects” means one or more of the objects.

In the following description, some embodiments of the present invention may be implemented at least in part by a data processing device system configured by a software program. Such a program may equivalently be implemented as multiple programs, and some or all of such software program(s) may be equivalently constructed in hardware.

Further, the phrase “at least” is or may be used herein at times merely to emphasize the possibility that other elements may exist beside those explicitly listed. However, unless otherwise explicitly noted (such as by the use of the term “only”) or required by context, non-usage herein of the phrase “at least” nonetheless includes the possibility that other elements may exist besides those explicitly listed. For example, the phrase ‘based at least on A’ includes A as well as the possibility of one or more other additional elements besides A. In the same manner, the phrase ‘based on A’ includes A, as well as the possibility of one or more other additional elements besides A. However, the phrase ‘based only on A’ includes only A. Similarly, the phrase ‘configured at least to A’ includes a configuration to perform A, as well as the possibility of one or more other additional actions besides A. In the same manner, the phrase ‘configured to A’ includes a configuration to perform A, as well as the possibility of one or more other additional actions besides A. However, the phrase ‘configured only to A’ means a configuration to perform only A.

The word “device”, the word “machine”, the word “system”, and the phrase “device system” all are intended to include one or more physical devices or sub-devices (e.g., pieces of equipment) that interact to perform one or more functions, regardless of whether such devices or sub-devices are located within a same housing or different housings. However, it may be explicitly specified according to various embodiments that a device or machine or device system resides entirely within a same housing to exclude embodiments where the respective device, machine, system, or device system resides across different housings. The word “device” may equivalently be referred to as a “device system” in some embodiments.

Further, the phrase “in response to” may be used in this disclosure. For example, this phrase may be used in the following context, where an event A occurs in response to the occurrence of an event B. In this regard, such phrase includes, for example, that at least the occurrence of the event B causes or triggers the event A.

The phrase “derivative thereof” and the like is or may be used herein at times in the context of a derivative of data or information merely to emphasize the possibility that such data or information may be modified or subject to one or more operations. For example, if a device generates first data for display, the process of converting the generated first data into a format capable of being displayed may alter the first data. This altered form of the first data may be considered a derivative of the first data. For instance, the first data may be a one-dimensional array of numbers, but the display of the first data may be a color-coded bar chart representing the numbers in the array. For another example, if the above-mentioned first data is transmitted over a network, the process of converting the first data into a format acceptable for network transmission or understanding by a receiving device may alter the first data. As before, this altered form of the first data may be considered a derivative of the first data. For yet another example, generated first data may undergo a mathematical operation, a scaling, or a combining with other data to generate other data that may be considered derived from the first data. In this regard, it can be seen that data is commonly changing in form or being combined with other data throughout its movement through one or more data processing device systems, and any reference to information or data herein is intended to include these and like changes, regardless of whether or not the phrase “derivative thereof” or the like is used in reference to the information or data, unless otherwise required by context. As indicated above, usage of the phrase “or a derivative thereof” or the like merely emphasizes the possibility of such changes. Accordingly, the addition of or deletion of the phrase “or a derivative thereof” or the like should have no impact on the interpretation of the respective data or information. For example, the above-discussed color-coded bar chart may be considered a derivative of the respective first data or may be considered the respective first data itself.

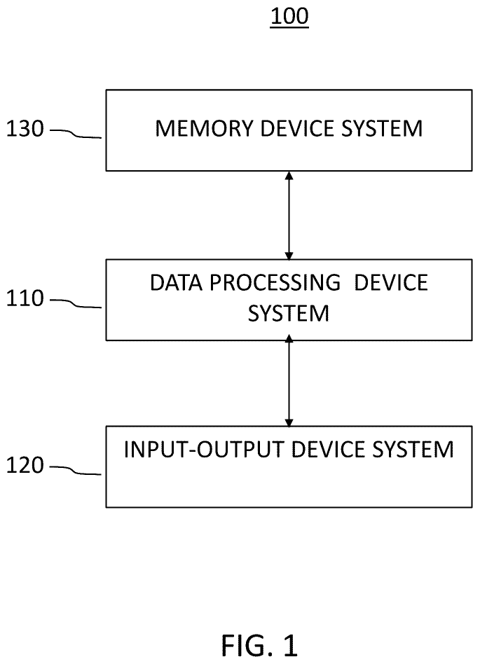

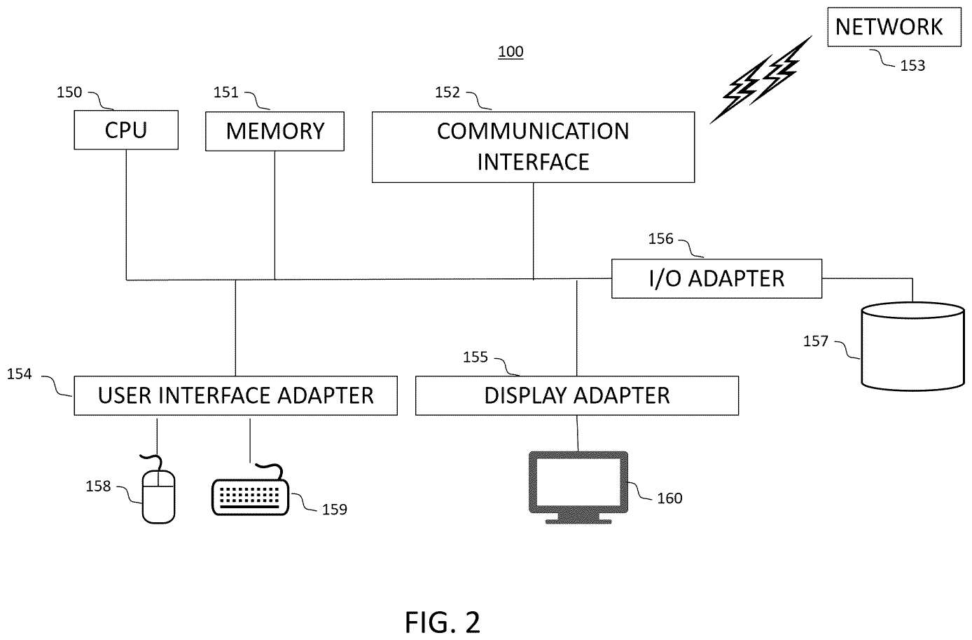

The term “program” in this disclosure should be interpreted to include one or more programs including as a set of instructions or modules that may be executed by one or more components in a system, such as a controller system or data processing device system, in order to cause the system to perform one or more operations. The set of instructions or modules may be stored by any kind of memory device, such as those described subsequently with respect to the memory device system 130, 151, or both, shown in FIGS. 1 and 2, respectively. In addition, this disclosure may describe or similarly describe that the instructions or modules of a program are configured to cause the performance of an action. The phrase “configured to” in this context is intended to include at least (a) instructions or modules that are presently in a form executable by one or more data processing devices to cause performance of the action (e.g., in the case where the instructions or modules are in a compiled and unencrypted form ready for execution), and (b) instructions or modules that are presently in a form not executable by the one or more data processing devices, but could be translated into the form executable by the one or more data processing devices to cause performance of the action (e.g., in the case where the instructions or modules are encrypted in a non-executable manner, but through performance of a decryption process, would be translated into a form ready for execution). Such descriptions should be deemed to be equivalent to describing that the instructions or modules are configured to cause the performance of the action. The word “module” may be defined as a set of instructions. The word “program” and the word “module” may each be interpreted to include multiple sub-programs or multiple sub-modules, respectively. In this regard, reference to a program or a module may be considered to refer to multiple programs or multiple modules.

Further, it is understood that information or data may be operated upon, manipulated, or converted into different forms as it moves through various devices or workflows. In this regard, unless otherwise explicitly noted or required by context, it is intended that any reference herein to information or data includes modifications to that information or data. For example, “data X” may be encrypted for transmission, and a reference to “data X” is intended to include both its encrypted and unencrypted forms, unless otherwise required or indicated by context. However, non-usage of the phrase “or a derivative thereof” or the like nonetheless includes derivatives or modifications of information or data just as usage of such a phrase does, as such a phrase, when used, is merely used for emphasis.

Further, the phrase “graphical representation” used herein is intended to include a visual representation presented via a display device system and may include computer-generated text, graphics, animations, or one or more combinations thereof, which may include one or more visual representations originally generated, at least in part, by an image-capture device.

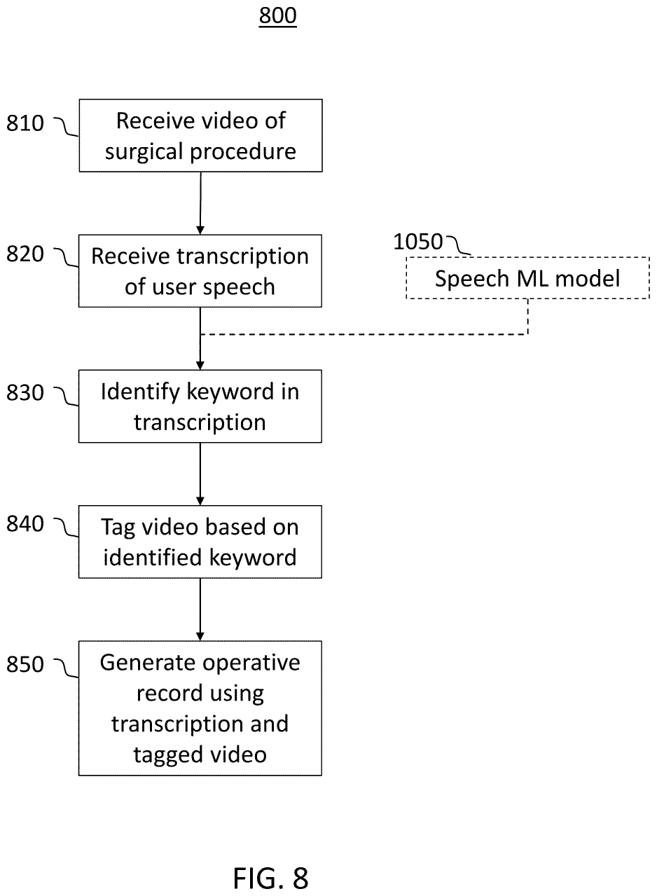

Further still, example methods are described herein with respect to FIGS. 8-11, 13, 14 and 16-18. Such figures are described to include blocks associated with computer-executable instructions. It should be noted that the respective instructions associated with any such blocks herein need not be separate instructions and may be combined with other instructions to form a combined instruction set. The same set of instructions may be associated with more than one block. In this regard, the block arrangement shown in method FIGS. 8-11, 13, 14 and 16-18 herein is not limited to an actual structure of any program or set of instructions or required ordering of method tasks, and such method FIGS. 8-11, 13, 14 and 16-18, according to some embodiments, merely illustrates the tasks that instructions are configured to perform, for example upon execution by a data processing device system in conjunction with interactions with one or more other devices or device systems.

FIG. 1 schematically illustrates a system 100 according to some embodiments. In some embodiments, the system 100 may be a computing device 100 (as shown in FIG. 2). In some embodiments, the system 100 includes a data processing device system 110, an input-output device system 120, and a processor-accessible memory device system 130. The processor-accessible memory device system 130 and the input-output device system 120 are communicatively connected to the data processing device system 110.

The data processing device system 110 includes one or more data processing devices that implement or execute, in conjunction with other devices, such as one or more of those in the system 100, control programs associated with some of the various embodiments. Each of the phrases “data processing device”, “data processor”, “processor”, and “computer” is intended to include any data processing device, such as a central processing unit (“CPU”), a desktop computer, a laptop computer, a mainframe computer, a tablet computer, a personal digital assistant, a cellular phone, and any other device configured to process data, manage data, or handle data, whether implemented with electrical, magnetic, optical, biological components, or other.

The memory device system 130 includes one or more processor-accessible memory devices configured to store information, including the information needed to execute the control programs associated with some of the various embodiments. The memory device system 130 may be a distributed processor-accessible memory device system including multiple processor-accessible memory devices communicatively connected to the data processing device system 110 via a plurality of computers and/or devices. On the other hand, the memory device system 130 need not be a distributed processor-accessible memory system and, consequently, may include one or more processor-accessible memory devices located within a single data processing device.

Each of the phrases “processor-accessible memory” and “processor-accessible memory device” is intended to include any processor-accessible data storage device, whether volatile or nonvolatile, electronic, magnetic, optical, or otherwise, including but not limited to, registers, floppy disks, hard disks, Compact Discs, DVDs, flash memories, ROMs, and RAMs. In some embodiments, each of the phrases “processor-accessible memory” and “processor-accessible memory device” is intended to include a non-transitory computer-readable storage medium. In some embodiments, the memory device system 130 can be considered a non-transitory computer-readable storage medium system.

The phrase “communicatively connected” is intended to include any type of connection, whether wired or wireless, between devices, data processors, or programs in which data may be communicated. Further, the phrase “communicatively connected” is intended to include a connection between devices or programs within a single data processor, a connection between devices or programs located in different data processors, and a connection between devices not located in data processors at all. In this regard, although the memory device system 130 is shown separately from the data processing device system 110 and the input-output device system 120, one skilled in the art will appreciate that the memory device system 130 may be located completely or partially within the data processing device system 110 or the input-output device system 120. Further in this regard, although the input-output device system 120 is shown separately from the data processing device system 110 and the memory device system 130, one skilled in the art will appreciate that such system may be located completely or partially within the data processing system 110 or the memory device system 130, depending upon the contents of the input-output device system 120. Further still, the data processing device system 110, the input-output device system 120, and the memory device system 130 may be located entirely within the same device or housing or may be separately located, but communicatively connected, among different devices or housings. In the case where the data processing device system 110, the input-output device system 120, and the memory device system 130 are located within the same device, the system 100 of FIG. 1 can be implemented by a single application-specific integrated circuit (ASIC) in some embodiments.

The input-output device system 120 may include a microphone, a speaker, a mouse, a keyboard, a touch screen, another computer, or any device or combination of devices from which a desired selection, desired information, instructions, or any other data is input to the data processing device system 110. The input-output device system 120 may include any suitable interface for receiving information, instructions or any data from other devices and systems described in various ones of the embodiments.

The input-output device system 120 also may include an image generating device system, a display device system, a speaker device system, a processor-accessible memory device system, or any device or combination of devices to which information, instructions, or any other data is output from the data processing device system 110. In this regard, if the input-output device system 120 includes a processor-accessible memory device, such memory device may or may not form part or all of the memory device system 130. The input-output device system 120 may include any suitable interface for outputting information, instructions or data to other devices and systems described in various ones of the embodiments. In this regard, the input-output device system may include various other devices or systems described in various embodiments.

FIG. 2 shows an example of a computing device system 100, according to some embodiments. The computing device system 100 may include a processor 150, corresponding to the data processing device system 110 of FIG. 1, in some embodiments. The memory 151, input/output (I/O) adapter 156, and non-transitory storage medium 157 may correspond to the memory device system 130 of FIG. 1, according to some embodiments. The user interface adapter 154, mouse 158, keyboard 159, display adapter 155, and display 160 may correspond to the input-output device system 120 of FIG. 1, according to some embodiments. The computing device 100 may also include a communication interface 152 that connects to a network 153 for communicating with other computing devices 100.

Further, it is understood that information or data may be operated upon, manipulated, or converted into different forms as it moves through various devices or workflows. In this regard, unless otherwise explicitly noted or required by context, it is intended that any reference herein to information or data includes modifications to that information or data. For example, “data X” may be encrypted for transmission, and a reference to “data X” is intended to include both its encrypted and unencrypted forms. For another example, “image information Y” may undergo a noise filtering process, and a reference to “image information Y” is intended to include both the pre-processed form and the noise-filtered form. In other words, both the pre-processed form and the noise-filtered form are considered to be “image information Y”. In order to stress this point, the phrase “or a derivative thereof” or the like may be used herein. Continuing the preceding example, the phrase “image information Y or a derivative thereof” refers to both the pre-processed form and the noise-filtered form of “image information Y”, with the noise-filtered form potentially being considered a derivative of “image information Y”. However, non-usage of the phrase “or a derivative thereof” or the like nonetheless includes derivatives or modifications of information or data just as usage of such a phrase does, as such a phrase, when used, is merely used for emphasis.

Figs. FIGS. 8-11, 13, 14 and 16-18 include data generation and flow diagrams, which may implement various embodiments of methods 800-1100, 1300, 1400, and 1600-1800 by way of associated computer-executable instructions according to some example embodiments. In various example embodiments, a memory device system (e.g., memory device system 130) is communicatively connected to a data processing device system (e.g., data processing device systems 110, otherwise stated herein as “e.g., 110”) and stores a program executable by the data processing device system to cause the data processing device system to execute various embodiments of methods 800-1100, 1300, 1400, and 1600-1800 via interaction with at least, for example, various databases. In these various embodiments, the program may include instructions configured to perform, or cause to be performed, various ones of the instructions associated with execution of various embodiments of methods 800-1100, 1300, 1400, and 1600-1800. In some embodiments, methods 800-1100, 1300, 1400, and 1600-1800 may include a subset of the associated blocks or additional blocks than those shown, respectively, in FIGS. 8-11, 13, 14 and 16-18. In some embodiments, methods 800-1100, 1300, 1400, and 1600-1800 may include a different sequence indicated between various ones of the associated blocks shown, respectively, in FIGS. 8-11, 13, 14 and 16-18.

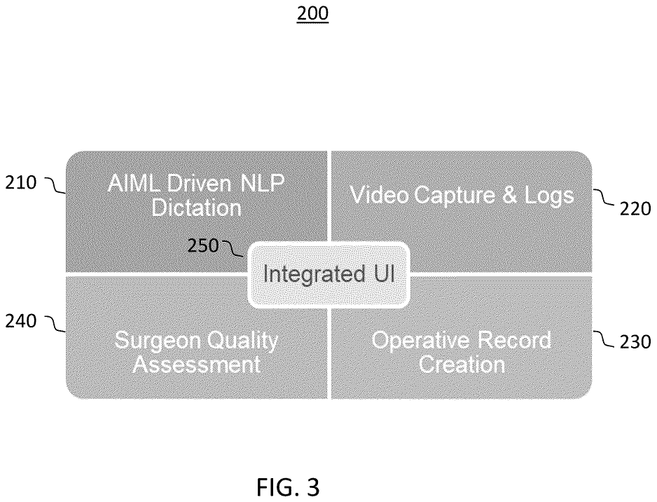

According to some embodiments of the present invention, the system 100 includes some or all of an operative report generation system 200 shown in FIG. 3, or vice versa. In this regard, FIG. 3 illustrates an operative report generation system 200, according to some embodiments of the present invention. The operative report generation system 200 may be a particular implementation of the system 100, according to some embodiments.

In some embodiments of the invention, the operative report generation system 200 includes a dictation module 210, a video and image capture logging module 220 (including an image processing module), an operative record generation module 230, a surgeon quality assessment and review module 240, and a user interface module 250. In some embodiments of the invention, the dictation module 210 is based on natural language processing methodologies and implemented using artificial intelligence and machine learning techniques. In some embodiments of the invention, video and image capture logging module 220 records video of the surgical operation, permits capturing of video frames as images, and tagging of video clips and images with descriptive information. The operative record generation module 230 captures information from the dictation module 210 and the video and image capture logging module 220 and integrates the information into an operative record 600 that can then be edited by the surgeon. This enables real-time operative record creation and editing, and the final operative record 600 can then be stored securely in the healthcare provider environment. The surgeon quality assessment module 240 enables extraction of information from the operative record 600 on a partial or a whole basis to be shared with a variety of stakeholders. An AI based scoring system is used as part of the surgeon quality assessment module 240 to determine ratings. Ratings are stored securely, and a surgeon rating report can be generated as a CSV, word, pdf or XML file, which can be printed and/or shared with other systems through defined API's.

Machine learning-based dictation processing, performed in the dictation module 210, involves using a computer program to train a speech transcription model (machine learning model) 1050, which recognizes patterns in audio recordings, to transcribe spoken words into written text. The process typically involves collecting large amounts of audio recordings to be used as training data for the speech transcription model, processing the audio recordings to extract features such as the frequency of different sounds and the duration of pauses, and training a machine learning model, such as a deep neural network, using the extracted features to recognize patterns in the audio and generate text output. The trained speech transcription model 1050 is tested on a separate dataset to evaluate its accuracy and make any necessary adjustments to ensure it is generalized. Once the speech transcription model 1050 is deemed accurate enough, it can be deployed to transcribe new audio recordings in real-time.

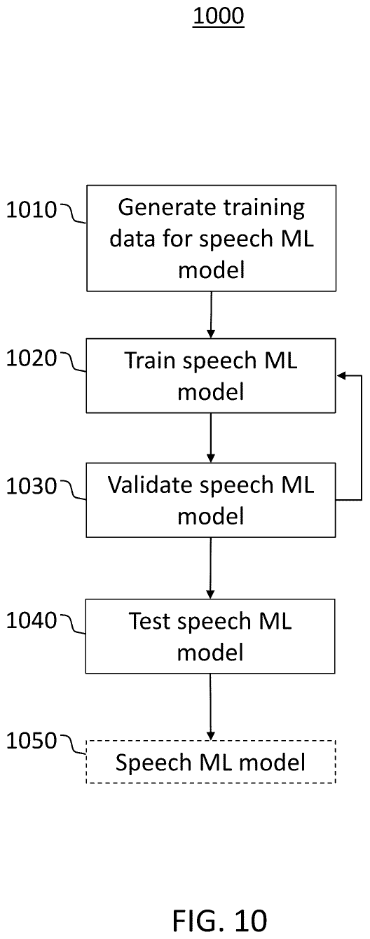

FIG. 10 shows a flowchart for an exemplar method 1000 of generating the speech transcription model 1050. In the training phase, in step 1010, large amounts of audio recordings are collected and used as training data for the speech transcription model. The audio can come from various sources, such as user recordings or pre-recorded audio datasets. The audio recordings are transcribed by humans or by using other speech recognition software to create labeled datasets. In step 1020, the audio data is preprocessed to extract relevant features such as Mel-Frequency Cepstral Coefficients (MFCCs), spectrograms, and other acoustic features. A machine learning model is chosen based on the specific requirements of the application. Popular models for speech recognition include Convolutional Neural Networks (CNNs) and Recurrent Neural Networks (RNNs). The labeled audio data is used to train the selected machine learning model. The machine learning model learns from the input audio and associated transcription examples, iteratively updating its internal parameters to improve its performance.

In some embodiments, in steps 1030 and 1040, validation and testing of the trained model is performed to ensure that the model is generalized (it is not overfitted to the training data and can provide similar performance on new data as on the training data). In some embodiments, a portion of the collected training data is held back from the training set for validation (validation dataset) and testing (testing dataset). In step 1030, the validation dataset is used to estimate the trained model's performance while tuning the model's parameters to improve its accuracy. This validation step 1030 is performed iteratively with the training step 1020, to adjust the parameters of the trained model until a predetermined condition (for example, an error between the two sets being less than a predefined threshold) is met. The test dataset is used to generate an unbiased estimate of the performance of the final trained and validated model in step 1040. It is well known that evaluating the trained model using the training set would result in a biased score as the trained model is, by design, built to learn the biases in the training set. Thus, to evaluate the performance of a trained machine learning model, one needs to use data that has not been used for training.

In one embodiment, the collected audio recordings can be divided equally between the training set and the testing set. The machine learning model is trained using the training set and its performance is evaluated using the testing set. The machine learning model is considered to be generalized or well-trained if its performance on the testing set is within a desired range (error) of the performance on the training set. If the performance on the testing set is worse than the training set (the difference in error between the training set and the testing set is greater than a predefined threshold), a two-stage validation and testing approach may be used. The model's accuracy (error) is measured using standard metrics such as Word Error Rate (WER), Character Error Rate (CER), and Sentence Error Rate (SER).

In some embodiments, in a two-stage validation and testing approach, the collected audio recordings are divided between the training set, the validation set, and the testing set. The machine learning model is first trained using the training set, then its parameters are adjusted to improve the model's accuracy and generalization using the validation set, and, finally, the trained machine learning model is tested using the testing set.

In some embodiments, the data set may be divided equally between the desired training, validation, or testing sets. This works well when there is a large collection of data to draw from. In cases where the collection of data samples is limited, other well-known techniques, such as leave one out cross validation and testing or k-fold cross validation may be used to perform validation and testing. Cross-validation is a resampling procedure used to evaluate machine learning models on a limited data sample. The procedure has a single parameter called k that refers to the number of groups that a given data set is to be split into. As such, the procedure is often called k-fold cross-validation. When a specific value for k is chosen, such as k=10, the procedure becomes 10-fold cross-validation.

Cross-validation is primarily used to estimate how the trained model is expected to perform in general when used to make predictions on data not used during the training of the model. The dataset is shuffled randomly and divided into a predefined number (k) of groups. The training and testing process is performed k times, with one of the groups of data being held out as the testing set for each iteration and the remaining k-1 groups being used as the training set. Each model is fitted (trained) on the training set and evaluated (tested) on the test set to determine the level of generalization of the trained models.

Cross validation can help determine the model structure and the parameter training process for the machine learning model. For example, a neural network model can have one or more “hidden” layers of neurons between the input layer and the output layer. Further, different neural network models can be built with different numbers of neurons in the hidden layers and the output layers. In some embodiments, in the training phase, a plurality of machine learning models, such as neural network models having different numbers of layers and different numbers of neurons in each layer, are generated. Each of the plurality of machine learning models is trained using k-fold cross validation. The model (number of layers and number of neurons in each layer) having the highest predictive score is selected.

It is obvious to one of ordinary skill in the art that the machine learning model is not limited to neural networks, and other machine learning models, such as a Markov random field network, support vector machine, random forest of decision trees, or k-nearest neighbor, or a combination of different types of machine learning models may be used as the machine learning model for the speech transcription model 1050 in the dictation module 210.

In some embodiments, the video and image capture logging module 220 automatically extracts and tags (annotates) salient video clips and images of the surgical procedure for inclusion in the operative report. As used herein, ‘operative record’ and ‘operative report’ may be used interchangeably. In some embodiments of the invention, the video and image capture logging module 220 receives the written text (transcribed speech) from the dictation module 210 in real time, identifies keywords in the written text, and extracts and annotates video clips or images from a real time video feed of the surgery based on the identified keywords. The annotations may include a time stamp or the keywords that triggered the capturing of the video clips or images.

In some embodiments, the keywords may be predefined commands—such as “take snapshot”, “begin clip” and “end clip”—that explicitly instruct the video and image capture logging module 220 to capture a video clip or an image. When the surgeon wants the operative report generation system 200 to record an image, they can utter the words “take snapshot”. The dictation module 210 transcribes the speech into text in real time. The video and image capture logging module 220 receives the written text corresponding to the surgeon's dictation, recognizes the predefined keyword “take snapshot”, accesses a video feed of the surgery, and records an image at the time corresponding to the dictated command (“take snapshot”) by the surgeon. The processing happens in real time, which means the snapshot from the video feed is captured instantaneously with the utterance of the command by the surgeon. The video feed can also have a “preview” window where the past few seconds of video feed are recorded, to ensure that the appropriate video clip or image, at the time the command was given, is recorded. Similarly, the surgeon can use the verbal commands “begin clip” and “end clip” to instruct the operative report generation system 200 to record a video clip of a salient portion of the surgery. The video and image capture logging module 220 records and stores the video clips and images based on the dictated commands in a storage associated with the operative report generation system 200. The operative record generation module 230 accesses the stored video clips and images for generating the operative record 600.

In some embodiments, the keywords may be obtained from predefined templates associated with various surgical procedures. For example, an operative record template for an appendectomy may include keywords or key phrases that describe salient aspects of the surgical procedure. For example, the surgeon may utter the phrase “suprapubic port” when placing a port into the patient's suprapubic region, the phrases “visualizing mesoappendix” and “grasping mesoappendix” when they can see the appendix through a scope and grasp the appendix with forceps, “removing appendix” when they cut the appendix and place it in an endobag, etc. As with the previous embodiments, the dictation module 210 transcribes the speech into text in real time. The system 200 can use these keywords and phrases to automatically record video clips and images, from the video feed of the surgery. This allows a more natural feel during the surgery, where the surgeon can dictate what they are doing rather than commands for instructing the system 200 to record video clips or images. The video and image capture logging module 220 records and stores the video clips and images based on the recognized keywords or phrases in the storage associated with the operative report generation system 200. The operative record generation module 230 accesses the stored video clips and images for generating the operative record 600.

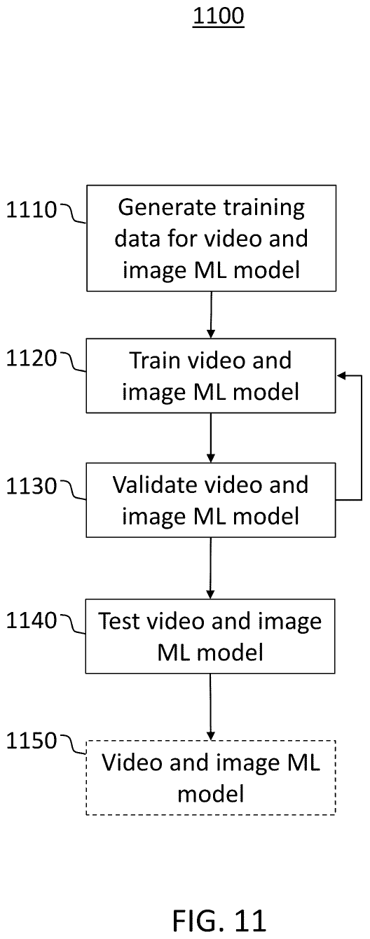

FIG. 11 shows a flowchart for an exemplar method 1100 of generating a video and image extraction machine learning model 1150. In some embodiments, the video and image capture logging module 220 includes a video and image extraction machine learning model 1150 that is trained on a large collection of annotated operative reports, to automatically capture video clips and images from the video feed of the surgery without user prompt or intervention. Similar machine learning techniques and models, as those described above with respect to the dictation module 210, may be used to train the video clip and image extraction model 1150 that recognizes salient portions of the video feed of the surgery, and automatically captures the video clips and images, even in a case where the surgeon does not provide any verbal commands, keywords, or phrases. In some embodiments, in step 1110, various visual features are computed from the video clips and images included in the collection of operative reports (the training data set). Commonly used types of features, extracted from images and videos, include color-based features, texture-based features, shape-based features, and motion-based features. Operative reports may include other features, besides the images and videos, that may be used to learn when the video and image capture logging module 220 should record images and videos, for example, timestamps and starting/ending of various procedures.

In some embodiments, in step 1120, the video and image features, and other features extracted from the operative records 600, are used as training data to train a machine learning model that can monitor the video feed of the surgery, recognize when the video feed is showing a salient portion of the surgery based on the trained model, and automatically record a video clip or an image. In steps 1130 and 1140, respectively, validation and testing of the trained video and image model 1150 is performed. The methods for training, validating, and testing the machine learning model, to be used as the video clip and image extraction model 1150, are similar to those used for training, validating, and testing the speech transcription machine learning model described above. In some embodiments, different video clip and image extraction models (machine learning models) may be trained for different types of surgeries, using only operative records 600 for the corresponding surgeries as training data, to provide increased accuracy and specificity for different surgery types. In some embodiments, a combination of a generalized model and surgery specific models may be used for extracting salient video clips and images.

In some embodiments, the video clip and image extraction model 1150 may be used in stand-alone mode, without receiving any transcription from the dictation module 210, to automatically extract salient video clips and images from the live feed of the surgery. In other embodiments, the video clip and image extraction model 1150 may work in conjunction with commands or phrases recognized by the dictation module 210 to augment the video clips and images recorded in response to the surgeon's dictation. The video and image capture logging module 220 may store video clips and images recorded based on transcribed speech with a different tag, or in a different location, than video clips and images automatically recorded using the trained video clip and image extraction model 1150.

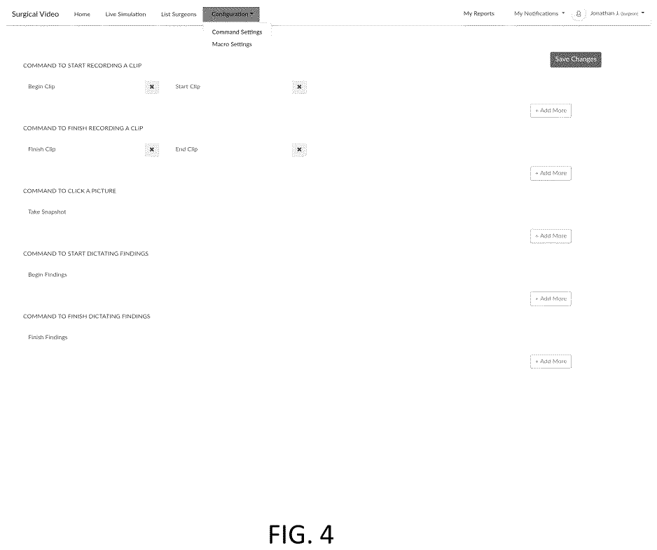

FIG. 4 shows an exemplar user interface screen, displayed on the user interface 250, for the operative report generation system 200, according to some embodiments of the present invention. In some embodiments, user interface screens can be used to set various features that customize the user's interactions with the hardware, the operating environment of the system 200, the manner of interacting with the system 200, the library of macros, the template for the operative report 600, the electronic medical record (EMR), and other metadata and media to be added to the operative report 600. In some embodiments, the customizable hardware interactions include system activation, voice-based interaction during procedure, system de-activation/pause/suspend mode, integration with multiple video and imaging sources, integration with robotic equipment, and integration with existing surgical environment/systems. The software environment customizations include user-based preferences, customizable templates for dictation and report generation, user voice recognition, user accent recognition and definition, and noise cancellation or disregarding of non-user voice/noise. The customizable commands include user preferred commands for system interaction and a standard library that can be modified. The customizable macro library includes user and surgery specific templates of macros and associated content, customizable macros and content based on institution or surgeon, ability to import macros and content.

In some embodiments, the operative report template can be customized to the institution. The library may include multiple templates that can be customized, for example, using logo/institution-based layout, to follow institution-based workflow/content flow customization, or support multiple output formats (csv, word, pdf, xml). The electronic medical record can be customized to include EMR specific API's to fetch data, EMR specific API's to transmit data and formatted content, and API management functions to allow for communication completion.

In some embodiments, the operative report generation system 200 can also include other media and metadata to enrich the surgeon's dictated notes. For example, the system 200 can be customized to permit input of various media within the operative report 600 and workflow, such as the ability to add images and snapshots to reports, the ability to add videos to the report 600, the ability to add voice content to the report 600. Other customizations include the ability to edit/manage report content, the ability to provide voice commands for report formatting and content input, support for multiple report formats available based on templates loaded into the system 200, the ability to send content and report to various sources, the ability to share the report 600 securely with other staff and third party systems and personnel, generating workflow for task management, and reporting status maintenance.

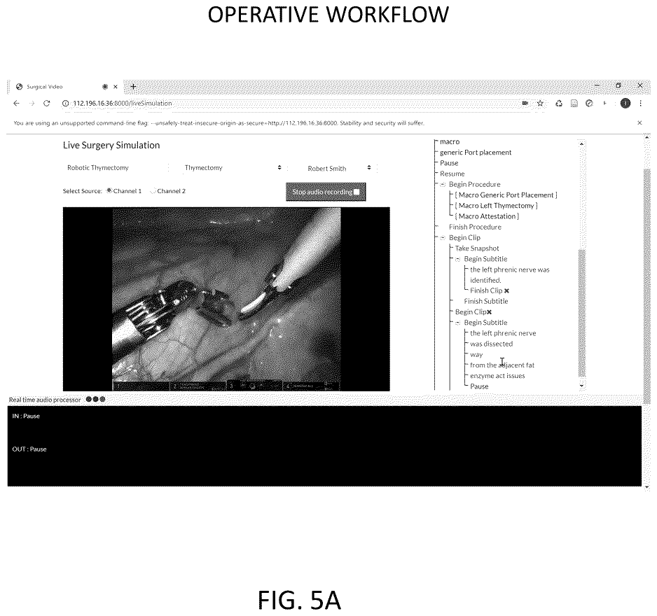

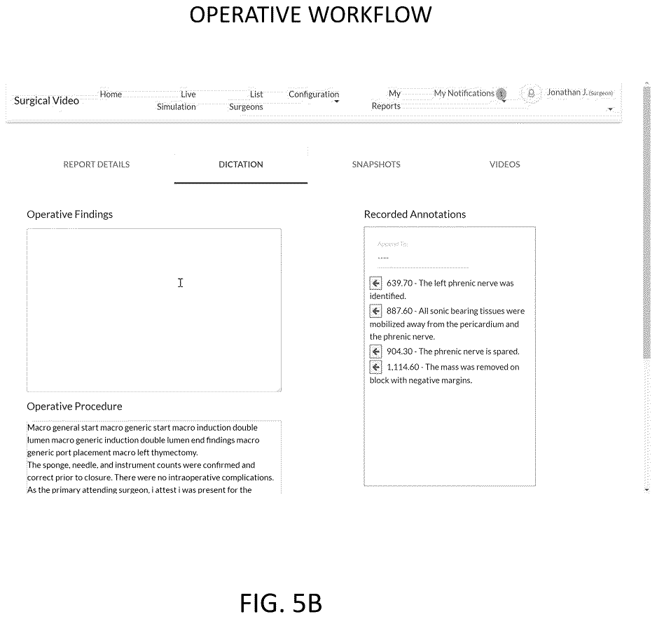

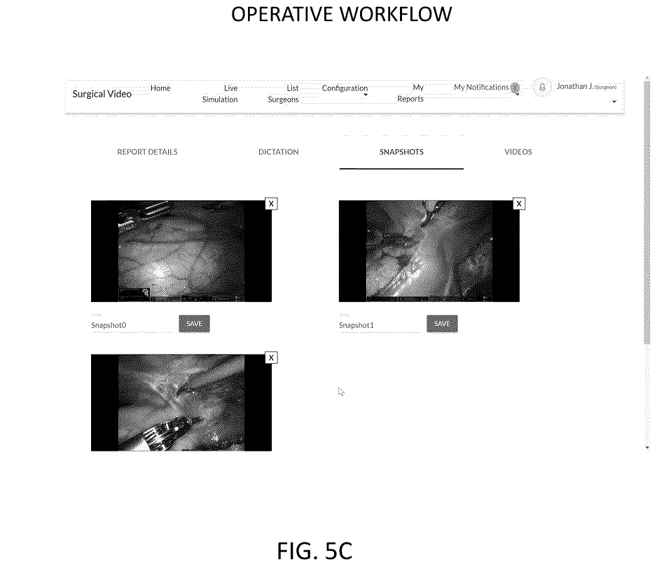

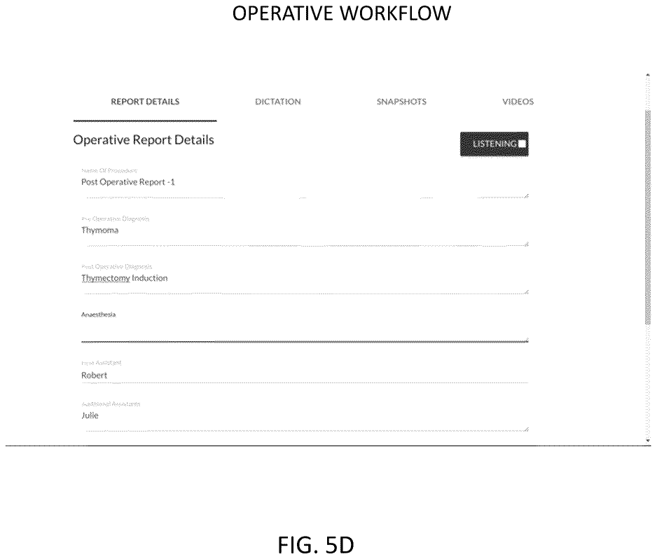

In some embodiments, the operative record generation module 230 performs an exemplar workflow, as shown in FIGS. 5A-5D, to generate the operative report 600. The workflow includes several steps. In a first step, a consent discussion with the patient is recorded. During the consent discussion, a full discussion of risks, benefits, and rationale of the intended procedure is conducted with the patient prior to starting the surgical procedure, and informed consent of the patient or their representative is obtained and recorded. In the next step, initial patient entry into the operative report 600 is performed. After the patient is brought into the surgical venue, the patient identity is confirmed. Other optional information, such as the placement of the patient on the operating table, and securing of the patient to the operating table, are recoded. For example, the patient may have been placed on the operating table in the supine position and all pressure points may have been padded. Serial compression devices may have been placed on the lower extremities. These observations are recorded during the initial patient entry step.

In some embodiments of the invention, in the next step, pre-operative procedures are recorded. For example, a patient may be administered with anesthesia, pre-operative antibiotics, or other medications. The site of the operation may be marked and confirmed. Availability of imaging studies and other diagnostic information required to perform the surgery may be confirmed and recorded.

Once pre-operative procedures are completed, recording of the surgery begins. In some embodiments of the invention, various surgical procedures may be recorded in the operative report generation system 200. In some embodiments of the invention, the operative report generation system 200 includes a plurality of templates associated with various surgical procedures. For example, a thymectomy induction template may be used to record a surgical procedure where, after induction of general endotracheal anesthesia, appropriate access and monitoring lines were placed by the anesthesia team, a double lumen bronchial blocker was placed and its position confirmed with fiberoptic bronchoscopy, and left lung isolation was instituted. As another example, a thymectomy positioning template may be used to record a surgical procedure where the patient was placed in the right hemi-lateral decubitus position with the left side up after confirming operative laterality, all appropriate pressure points were padded, and the patient was prepped and draped in the usual sterile fashion after marking incision sites. As another example, a port placement template may be used to record a surgical procedure where Incision sites were marked and the initial robotic port placed under direct vision with the optical separator trocar, pneumothorax was instituted with CO2 insufflation at a pressure of 8 mmHg, the remaining instrument ports were placed under direct vision, the robotic cart was brought in and attached to the ports, and all instruments were brought in under direct vision.

Upon completion of a surgical procedure, a verification and attestation step may be used to confirm that sponge, needle, and instrument counts were correct prior to closure and there were no intraoperative complications. The primary attending surgeon, or other physician(s), may attest that they were present for the entirety of the procedure and performed or directly supervised all key and non-key portions of the procedure.

The surgeon can also perform real-time editing and finalization of operation report 600. FIG. 5B shows an exemplar user interface screen for review and modification of the initial operative report 600. In some embodiments, the surgeon can review the operative report 600 while in progress or after completion of the surgical procedure. The surgeon can edit the report 600 through keyboard or voice command. Editing of content, images (captions as well as title) and video is also available through the system 200. The surgeon can rearrange, delete, or modify content, images, video and voice commands. The surgeon can also mark specific content as “private”, which will only be shared/viewed by specific users. User specific content access can be managed through defined roles and permissions.

In the final step, the generated report 600 is stored, along with annotated/tagged media such as text, voice, images and video, captured by the system 200 during dictation of the procedure by the surgeon. The final report 600, or specific images, videos, voice memo's can be sent to multiple sources or become part of a workflow to allow for securely sharing content.

In some embodiments, one or more of the workflow steps discussed above may be implemented as macros that can be executed using a simple or short command. This way, a surgeon performing the operation could use dictation “short cuts” to record an otherwise long procedure, thereby improving efficiency.

In some embodiments, natural language processing is used for voice capture and input. The workflow also includes capture and intelligent tagging of video and image during the surgical procedure. As discussed with reference to FIG. 4, the system allows hardware, security and workflow customization within an institution.

In some embodiments, video clip capture initiation and termination is managed through voice prompt/command, which is part of the command library. It allows for real-time command execution and timestamping the video clip and storing the exact clip into the system repository. This video clip can be “tagged” with content through voice dictation or keyboard. This can be done in real-time or after the fact.

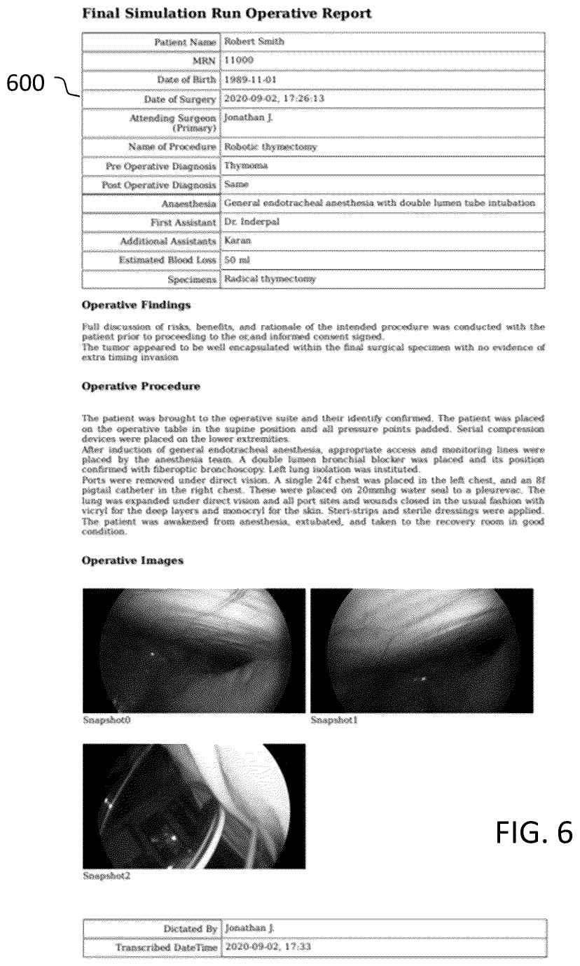

FIG. 6 shows an exemplar operative record 600 generated by the operative report generation system 200, according to some embodiments. The report 600 (operative record) is generated by collating all of the content, video, voice, and images in a specified format that is determined by the selected template in the system 200. The report generation process identifies “required” content and “optional” content, which is controlled by the surgeon and/or institution and set in the system preferences. The operative report 600 goes through a checklist of items, order of items, and format to generate the final report 600. Everything is timestamped and tagged to allow for validation of user and changes.

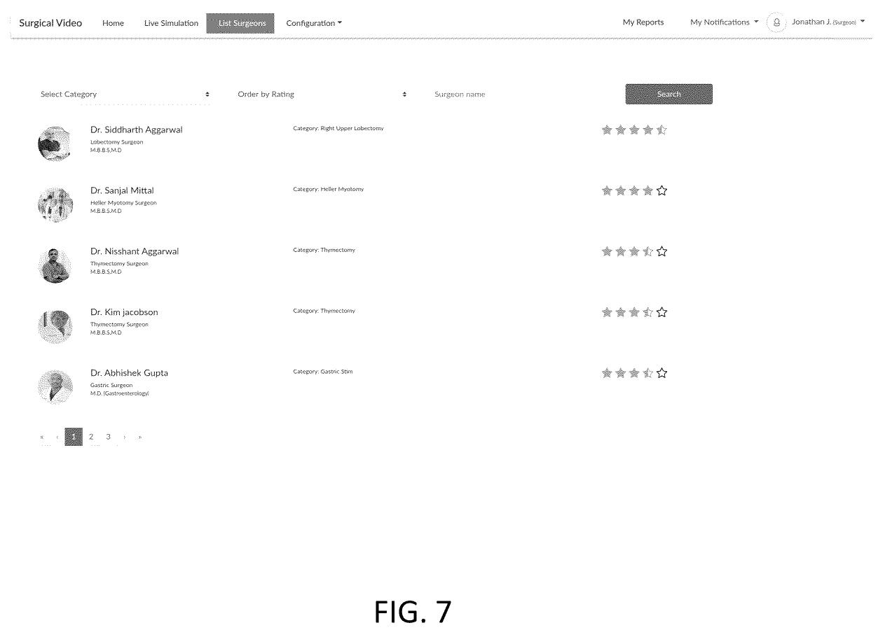

FIG. 7 shows an exemplar user interface 250 for assessing and managing credentials based on operative reports 600 generated by the operative report generation system 200. In some embodiments, video clips captured during a procedure are used to visually assess the surgeon's technical competency through a direct rating scale. These datapoints can used at the discretion of the hospital administration/or appropriate authority for credentialing and certification processes. Videos and associated dictation and voice memo's along with images can be shared with a learning management system or an assessment system. Different users have access to the learning management and assessment systems. In some embodiments, these users have the ability to view and rate the surgical procedure and surgeon competency. In some embodiments, an AI-based scoring system may be used to determine ratings. Ratings are stored securely and a rating report can be generated as a CSV, word, pdf or XML file, which can be printed and/or shared with another system through defined API's.

FIG. 8 shows a flowchart for an exemplar method 800 of generating an operative report 600, according to some embodiments. In step 810, a video of the surgical procedure is received in real time and displayed on the user interface 250 of the operative report generation system 200. In step 820, a transcription of the user's (surgeon) speech is received. In step 830, the transcription is analyzed in real time, using the speech ML model 1050, to identify keywords associated with macros and tasks. For example, a keyword in the transcription may be a short-hand for a longer transcript to be added to the operative report 600. As another example, a keyword may trigger capturing images or a video portion of the real time video being displayed on the user interface 250. In step 840, when a keyword that triggers image or video capture is identified in the transcription, the images or video portion is recorded and stored. The images or video portion may be tagged with a timestamp or other metadata extracted from the transcription. In step 850, the operative report 600 is generated using the operative report template, the transcription, and the tagged images or video portions.

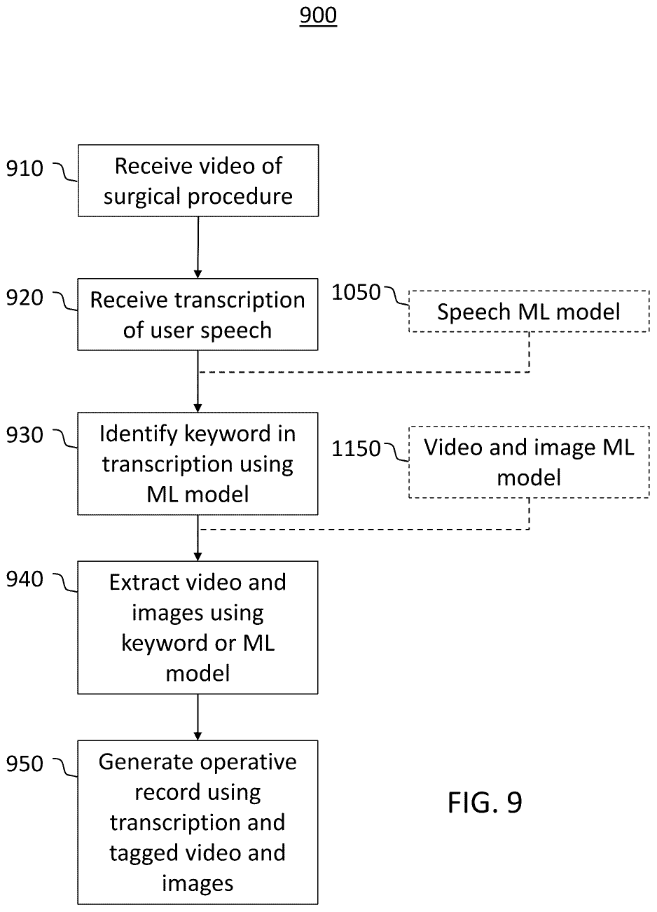

FIG. 9 shows a flowchart for another exemplar method 900 of generating an operative report 600, according to some embodiments. In step 910, a video of the surgical procedure is received in real time and displayed on the user interface 250 of the operative report generation system 200. In step 920, a transcription of the user's (surgeon) speech is received. In step 930, the transcription is analyzed in real time, using the speech ML model 1050, to identify keywords associated with macros and tasks. For example, a keyword in the transcription may be a short-hand for a longer transcript to be added to the operative report 600. As another example, a keyword may trigger capturing images or a video portion of the real time video being displayed on the user interface 250. In step 840, when either a keyword that triggers image or video capture is identified in the transcription or the video and image ML model 1150 determines that the video feed of the surgery is showing a salient aspect of the surgery, the images or video portion is recorded and stored. The images or video portion may be tagged with a timestamp or other metadata extracted from the transcription. In step 850, the operative report 600 is generated using the operative report template, the transcription, and the tagged images or video portions.

In some embodiments, the operative report generation system 200 includes a time-synchronization and alignment module (not illustrated) configured to synchronize (i) the surgical video stream, (ii) the audio stream, (iii) one or more transcriptions, (iv) device logs, and (v) user interactions, into a unified procedure timeline. In some embodiments, the time-synchronization and alignment module assigns a common time base to each data item using one or more of network time protocol synchronization, embedded timecode in the video stream, audio sample clocks, and monotonic clock timestamps generated at a capture interface. In some embodiments, the unified procedure timeline is stored as an index that maps time ranges to corresponding video segments, audio segments, transcription tokens, detected surgical events, and generated billing lines.

In some embodiments, the time-synchronization and alignment module compensates for transport latency and buffering delay by storing one or more offset parameters that are estimated during a calibration phase and/or continuously estimated during operation. In some embodiments, the calibration phase includes generating a known synchronization signal that is detectable in the audio stream and/or visible in the video stream, and computing an offset between an audio timestamp and a video timestamp in response to detecting the known synchronization signal.

In some embodiments, the operative report generation system 200 and the automated billing report generation system 300 maintain a set of related time-indexed data structures (also referred to herein as a data structure taxonomy) derived from the unified procedure timeline. In some embodiments, a unified monotonic procedure index comprises a core append-only time index in which each index entry includes a monotonic timestamp and one or more pointers to corresponding video segments, audio segments, transcription excerpts, detected events, and generated report element identifiers. In some embodiments, an evidence map comprises a materialized view derived from the unified monotonic procedure index and configured for report generation by providing, for each report element or billing line, a pre-resolved set of evidence links and time ranges without re-searching the underlying media streams. In some embodiments, an evidence attachment index comprises an export-packaging structure that maps report elements or billing lines to identifiers of attachable media objects, including clips, images, and transcription excerpts, together with corresponding time ranges and retrieval metadata for a destination system. In some embodiments, an auditable linkage record comprises a persisted billing-line-to-evidence mapping that associates a billing line identifier with at least one of a timestamp range, a video segment identifier, an audio segment identifier, or a transcription excerpt identifier, and is stored with version identifiers for applicable rule sets and phrase libraries to support repeatable downstream compliance review and audit.

In various embodiments, the operative report generation system 200 provides a technological improvement to intraoperative data acquisition, synchronization, and machine interpretation by implementing a bounded-latency multimodal processing architecture configured to deterministically align heterogeneous surgical data streams in real time. In some embodiments, the bounded-latency multimodal processing architecture operates during an active surgical workflow and performs live temporal normalization and cross-modal correlation among the surgical video stream, the audio stream, the transcription stream, device logs, and user interactions.

In some embodiments, unlike conventional post-procedural documentation systems that process recorded media asynchronously and later attempt alignment, the time-synchronization and alignment module performs alignment in-stream while data is being acquired. In some embodiments, in-stream alignment reduces computational drift associated with delayed or repeated alignment passes, reduces repeated random-access scanning operations over recorded media, and reduces redundant storage operations by enabling event-conditioned capture and indexing rather than indiscriminate recording of all media at full fidelity. In some embodiments, the time-synchronization and alignment module generates a unified monotonic procedure index that maps video frames, audio segments, transcription tokens or excerpts, detected events, and generated report elements to a common time base. In some embodiments, the unified monotonic procedure index comprises an append-only index structure in which each index entry includes a monotonic timestamp and one or more pointers to corresponding media segments, metadata records, model outputs, and/or report element identifiers.

In some embodiments, the time-synchronization and alignment module compensates for transport jitter, buffering latency, and device clock skew using one or more of latency estimation, clock offset estimation, drift tracking, and temporal interpolation. In some embodiments, latency estimation includes measuring one or more buffering delays at capture interfaces and associating a delay estimate with a corresponding stream segment. In some embodiments, temporal interpolation comprises computing, for at least one stream, an interpolated timestamp for a frame, sample window, or token boundary based on neighboring time-stamped samples and a stream-specific sampling rate.

In some embodiments, the time-synchronization and alignment module continuously refines one or more offset parameters during operation by evaluating cross-modal correlation signals. In some embodiments, cross-modal correlation signals include one or more of detected synchronization markers, detected co-occurring events observed in both video and audio, and alignment residuals derived from comparing expected versus observed temporal relationships between recognized events and narration. In some embodiments, responsive to detecting that an offset parameter exceeds a threshold, the time-synchronization and alignment module updates the offset parameter and records a versioned alignment state to preserve determinism and traceability.

In some embodiments, the unified monotonic procedure index improves computer functionality by enabling deterministic retrieval of multimodal evidence segments using a single temporal reference. In some embodiments, deterministic retrieval reduces random-access overhead by enabling retrieval operations to be performed using index lookups rather than repeated search operations across large video and audio objects. In some embodiments, deterministic retrieval improves evidentiary traceability by enabling each extracted media segment and each generated report element to be linked to a specific monotonic timestamp range, thereby improving downstream machine learning inference accuracy by reducing label noise and misalignment error in training and validation datasets.

In some embodiments, the operative report generation system 200 includes a streaming inference pipeline configured to perform real-time speech recognition and real-time event detection while reducing computational latency. In some embodiments, the streaming inference pipeline performs incremental decoding of the audio stream to produce partial transcriptions and updates the partial transcriptions as additional audio samples are received. In some embodiments, the streaming inference pipeline includes a bounded-latency buffer configured to allow capture of a pre-event window of video and/or audio in response to detecting a trigger condition, thereby improving capture fidelity when a trigger condition occurs between sampled frames or between transcription updates.

In some embodiments, the system maintains a bounded-latency rolling buffer of the surgical video stream configured to preserve a configurable pre-event interval. In some embodiments, the rolling buffer is implemented as a circular buffer storing a most recent window of frames or encoded segments having a predetermined maximum duration and a predetermined maximum memory size. In some embodiments, responsive to detecting a trigger condition, the system automatically commits buffered pre-event media corresponding to at least a portion of the configurable pre-event interval together with post-event media to persistent storage. In some embodiments, the trigger condition includes at least one of a recognized voice command, a recognized keyword or phrase in a transcription stream, a detected surgical event output by a machine learning model, a device-log event, or a user interface interaction.

In some embodiments, the rolling buffer and commit operation are configured to maintain deterministic memory utilization by bounding the rolling buffer size and by committing media segments using a fixed segmentation policy. In some embodiments, the fixed segmentation policy includes generating an evidence segment having (i) a pre-event segment length parameter and (ii) a post-event segment length parameter, and storing, for the evidence segment, a timestamp range in a unified procedure timeline such that the committed media is retrievable deterministically using a single temporal reference.

In some embodiments, the streaming inference pipeline used for real-time speech recognition and event detection in the operative record generation system is also used by the automated billing report generation system to generate the transcription stream for billing-report generation. In some embodiments, the same bounded-latency buffering, incremental decoding, and time-stamping mechanisms are reused such that partial transcriptions generated for billing purposes share a common time base with the surgical video stream and other data sources, thereby reducing duplicate computation and improving consistency between operative records and billing reports.

In some embodiments, the video and image capture logging module 220 includes a multi-stage salient media selector configured to reduce storage and compute load while maintaining clinically meaningful captures. In some embodiments, the multi-stage salient media selector includes a first stage configured to downsample frames and perform low-cost feature extraction, and a second stage configured to perform higher-cost inference on candidate segments selected by the first stage. In some embodiments, the multi-stage salient media selector performs one or more of keyframe selection, redundancy suppression, blur detection, occlusion detection, smoke or fog detection, and endoscope white-balance correction to improve downstream usability of captured media.

In some embodiments, the operative record generation module 230 includes a structured report assembler configured to generate the operative record 600 as a structured document comprising a set of sections and fields derived from a selected template, where each field is linked to one or more evidence items in the unified procedure timeline. In some embodiments, the structured report assembler stores, for each generated sentence or clause, a provenance record linking the sentence or clause to one or more of a transcription excerpt, a timestamp range, a macro invocation, and a media identifier.

In some embodiments, the operative report generation system 200 includes a completeness and consistency validator configured to identify missing, inconsistent, or low-confidence documentation in real-time during the procedure and/or after completion. In some embodiments, the completeness and consistency validator evaluates a template-specific checklist of required fields and determines a completeness score. In some embodiments, in response to determining that a required field is missing or that a field confidence score is below a threshold, the system 200 generates a minimal-interaction prompt configured for hands-free operation, where the prompt is configured to be answered by a short spoken confirmation, a spoken selection among a small set of options, or a single user interface selection.

In some embodiments, the dictation module 210 includes a speaker separation and speaker identification submodule configured to reduce transcription error in an operating room environment. In some embodiments, the speaker separation and speaker identification submodule performs one or more of noise suppression, dereverberation, voice activity detection, diarization, and speaker embedding comparison to preferentially transcribe a primary speaker associated with a surgeon profile while suppressing non-user speech.

In some embodiments, the operative report generation system 200 includes a minimal-interaction control interface configured to reduce surgeon interaction while maintaining deterministic capture behavior. In some embodiments, the control interface includes a wake word detector and a command grammar configured to recognize a limited command set that triggers capture, section transitions, and macro expansion. In some embodiments, the command grammar is personalized per user and stored as part of user settings such that different users can achieve comparable system control with fewer spoken syllables and fewer disambiguation prompts.

In some embodiments, the operative report generation system 200 includes an adaptive learning module configured to improve future performance based on post-procedure edits while maintaining traceability. In some embodiments, edits performed by the surgeon to the operative report 600 are captured as labeled correction data that associates (i) an original transcription segment, (ii) an edited segment, and (iii) a context window including preceding and following tokens. In some embodiments, the adaptive learning module uses the labeled correction data to generate one or more fine-tuning datasets and stores the datasets with user and procedure metadata for subsequent model improvement.

In some embodiments, the operative report generation system 200 includes a multi-source video integration module configured to combine multiple imaging sources into a unified capture space. In some embodiments, the multiple imaging sources include an endoscope feed, a room camera feed, a robotic console feed, an ultrasound feed, and a fluoroscopy feed. In some embodiments, the multi-source video integration module generates a source identifier for each frame and stores the source identifier in association with captured images and clips such that the operative report 600 (and a billing report 1500, described later in this disclosure) can link evidence to the correct source.

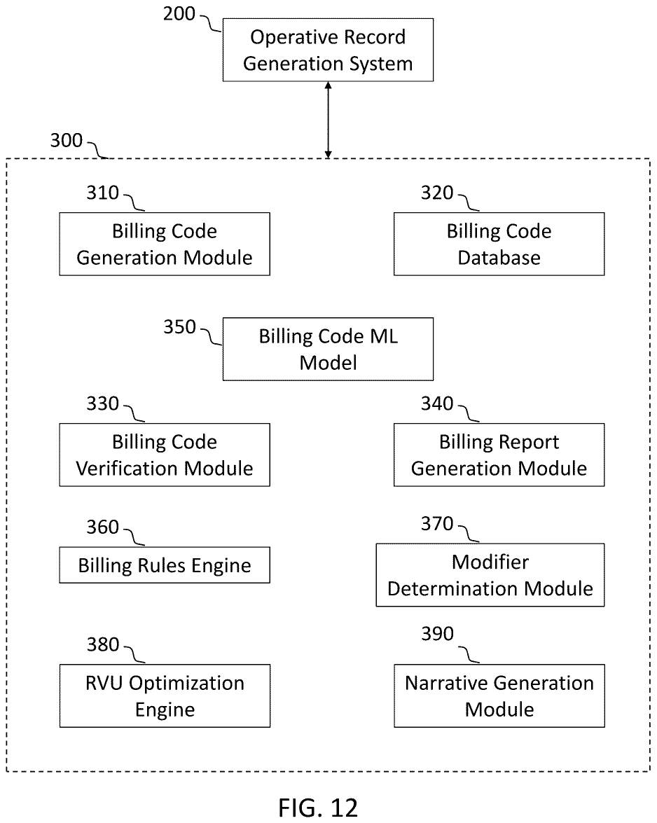

FIG. 12 illustrates an automated billing report generation system 300, integrated with the operative report generation system 200, according to some embodiments of the present invention. In some embodiments, the automated billing report generation system 300 includes one or more of a billing code generation module 310, a billing code database 320, a billing code verification module 330, a billing report generation module 340, a billing rules engine 360, a modifier determination module 370, a relative value unit (RVU) optimization engine 380, and a narrative generation module 390. The automated billing report generation system 300 is communicatively connected to the operative report generation system 200 and receives inputs from the dictation module 210, the video and image capture logging module 220, and the operative record generation module 230. In some embodiments, the billing report system 300 reuses the unified procedure timeline/evidence index, the transcription stream, and captured media identifiers from the operative report generation system 200, thereby reducing redundant computation and ensuring consistent timestamps/evidence anchors between the operative record 600 and billing report 1500.

In some embodiments, the billing code generation module 310 is configured to analyze at least one of the surgical video stream and the associated audio narration from the surgeon to identify billable events and to generate corresponding billing codes in real-time or near real-time. The billing code generation module 310 utilizes a billing code machine learning model 350 that has been trained to recognize surgical and other procedural billable events that are associated with specific billing codes.

The billing code database 320 stores a comprehensive collection of billing codes including, but not limited to, Medicare Benefits Schedule (MBS) codes, Current Procedural Terminology (CPT) codes, Healthcare Common Procedure Coding System (HCPCS) codes, International Classification of Diseases (ICD) codes for diagnoses and procedures, Diagnosis Related Group (DRG) codes, Revenue codes, and other standardized medical billing codes used by healthcare providers, insurance companies, and government healthcare programs. Each billing code in the billing code database 320 is associated with procedural descriptors, code definitions, relative value units (RVUs), global periods, modifier codes, and other billing-relevant metadata. In some embodiments, the RVU values stored in the billing code database 320 are accessed by the RVU optimization engine 380 to generate RVU-based validation outputs and evidence-anchored documentation opportunity records for use in billing report generation, as described later in this disclosure.

In some embodiments, the billing code database 320 is configured to receive periodic updates from external billing code authorities such as the American Medical Association (for CPT codes), the Centers for Medicare and Medicaid Services (for HCPCS and ICD codes), and other regulatory bodies, healthcare providers, insurance companies, and government healthcare programs. The periodic updates may include new billing codes, modifications to existing billing codes, deprecated codes, and changes to reimbursement rates. The billing code generation module 310 is configured to incorporate these updates into its processing logic, and the billing code machine learning model 350 may be retrained or fine-tuned based on the updated billing code information to maintain accuracy over time.

The billing code verification module 330 is configured to validate the billing codes generated by the billing code generation module 310 against the surgical video stream, the audio narration, and the operative record 600. In some embodiments, the billing code verification module 330 performs cross-referencing between the identified billable events, the generated billing codes, and established coding guidelines to identify potential coding errors, missing codes, or compliance issues. In some embodiments, the billing code verification module 330 may generate alerts or recommendations for human review when discrepancies are detected.

The billing report generation module 340 is configured to generate comprehensive billing reports that include the identified billing codes, timestamps associated with corresponding surgical events, confidence scores for each billing code, supporting evidence extracted from the surgical video stream and audio narration, and links to the relevant portions of the operative record 600. The billing reports may be formatted for integration with electronic health record (EHR) systems, practice management systems, claims submission systems, and other healthcare information technology infrastructure.

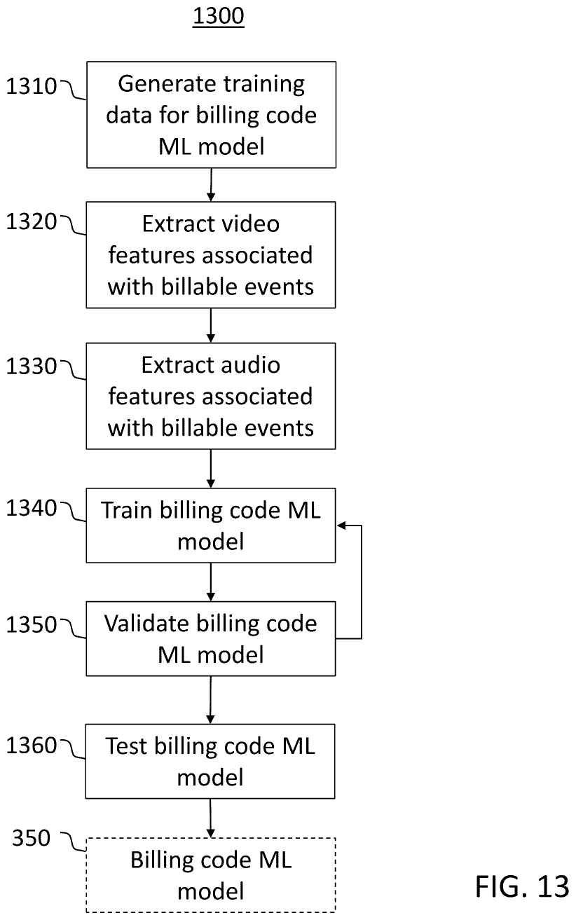

FIG. 13 shows a flowchart for an exemplar method 1300 of generating the billing code machine learning model 350, according to some embodiments. In step 1310, a plurality of training records is collected and assembled. Each training record in the ground truth dataset includes a surgical video recording of a completed surgical procedure, an audio narration associated with the surgical video (typically the surgeon's dictation during the procedure), expert identification of specific billable events within the surgical video with corresponding timestamps, and verified billing codes associated with each identified billable event.

The ground truth dataset represents a curated collection of historical surgical procedures where experienced medical coders or billing specialists have reviewed the surgical videos and audio narrations and have accurately assigned billing codes to each billable event. In some embodiments, the ground truth dataset includes surgical procedures from multiple surgical specialties, multiple surgeons, multiple healthcare facilities, and multiple payer types to ensure the billing code machine learning model 350 generalizes across diverse surgical contexts.

In step 1320, video features are extracted from the surgical videos in the training records. The video features may include visual representations of surgical instruments, anatomical structures, surgical actions (such as incisions, dissections, suturing, and cauterization), and other visually identifiable surgical events. In some embodiments, the video feature extraction utilizes computer vision techniques using convolutional neural networks (CNNs), object detection algorithms, action recognition models, and temporal modeling approaches such as recurrent neural networks (RNNs) or transformers.

In some embodiments, the video features extracted from the surgical video recordings include one or more of instrument features, anatomical structure features, motion features, or action recognition features. In some embodiments, instrument features identify the presence, type, or configuration of surgical tools in a frame or sequence of frames; anatomical structure features represent visual characteristics of organs, tissue planes, or pathological findings; motion features capture temporal changes between frames, including trajectories or velocities of instruments and anatomical structures; and action recognition features correspond to higher-level surgical actions, such as dissecting, suturing, clipping, stapling, cauterizing, or implant placement, recognized from sequences of frames and associated motion patterns.

In step 1330, audio features are extracted from the audio narrations in the training records. The audio features may include transcribed text of the surgeon's narration, identified keywords and phrases corresponding to specific surgical procedures and events, semantic analysis of the narration content, and temporal alignment between the audio narration and the surgical video. In some embodiments, the audio feature extraction utilizes natural language processing (NLP) techniques including speech-to-text transcription, named entity recognition, semantic parsing, and contextual language models.

In some embodiments, the audio-derived features include one or more of keyword features, medical entity features, semantic embedding features, or temporal alignment features between transcription excerpts and time ranges. In some embodiments, keyword features indicate the presence of predefined procedure-related terms, such as names of surgical steps, devices, or complications, detected within the transcription stream. In some embodiments, medical entity features are generated using named-entity recognition to identify and encode references to diagnoses, procedures, anatomical sites, medications, implants, or devices mentioned in the narration or transcription. In some embodiments, semantic embedding features are generated by a language model that maps words, phrases, or sentences from the transcription stream into vector representations capturing contextual meaning. In some embodiments, temporal alignment features associate each transcription excerpt or token with a corresponding timestamp or time range in the surgical video stream or audio stream, thereby enabling the billing code machine learning model to jointly reason over content and time.

In step 1340, the billing code machine learning model 350 is generated by training on the extracted video features and audio features to output billing codes corresponding to identified billable events. The training process learns the associations between visual and audio patterns in surgical procedures and the corresponding billing codes (ground truth) in the training data. In some embodiments, the billing code machine learning model 350 is implemented as a multimodal neural network that fuses video and audio features to make billing code predictions. The model architecture may include attention mechanisms to focus on relevant portions of the surgical video and audio narration, hierarchical representations to capture both local surgical actions and overall procedure context, and output layers configured to predict multiple billing codes with associated confidence scores.

In steps 1350 and 1360, validation and testing of the trained billing code machine learning model 350 is performed to ensure that the model generalizes to new surgical procedures not seen during training. The validation and testing methodology follows similar principles as described above for the speech transcription model 1050 and the video and image extraction model 1150, including separation of training, validation, and testing datasets, hyperparameter tuning, and performance evaluation using metrics such as precision, recall, F1 score, and area under the receiver operating characteristic curve (AUC-ROC) for billing code prediction accuracy.

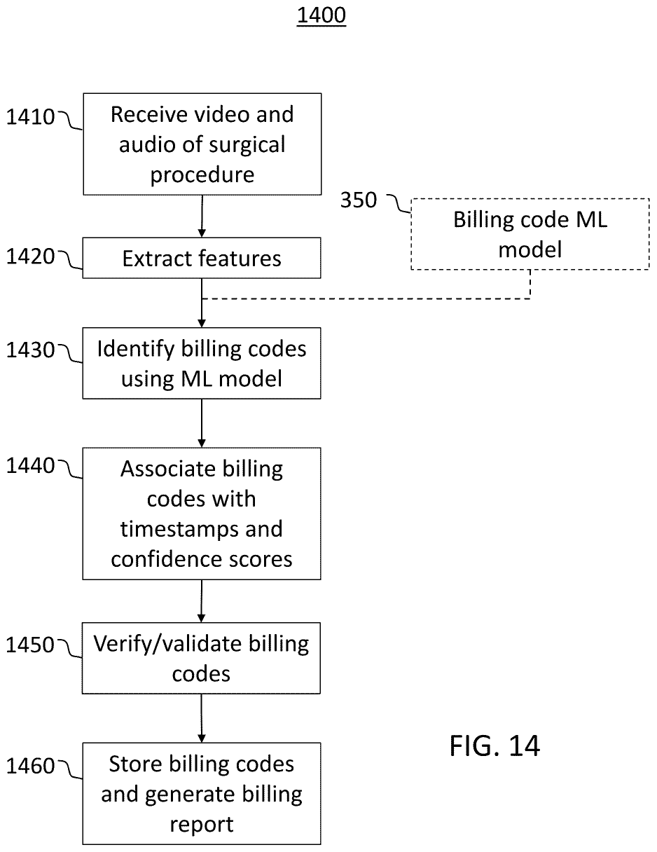

FIG. 14 shows a flowchart for an exemplar method 1400 of automatically generating billing codes for a surgical procedure, according to some embodiments. In step 1410, at least one of the surgical video stream and associated audio narration are received in real-time during the surgical procedure. In step 1420, features are extracted from at least one of the surgical video stream and the associated audio narration using the same feature extraction pipelines used during training. In step 1430, the extracted features are input into the trained billing code machine learning model 350 to output one or more billing codes for the surgical procedure. In step 1440, the output billing codes are associated with corresponding timestamps and confidence scores. In step 1450, the billing codes are verified against the operative record 600 and billing code guidelines. In step 1460, the verified billing codes are stored in association with the surgical procedure and the operative record 600 and a billing report 1500 is generated.

In some embodiments, the automated billing code generation occurs in real-time during the surgical procedure, enabling the surgeon and support staff to receive immediate feedback on billable events as they occur. This real-time billing code generation provides several advantages over conventional post-procedure billing approaches.

First, real-time billing code generation improves accuracy by capturing billing-relevant events as they occur, when the context is freshest and most accurately represented in the video and audio streams. The surgeon is provided with immediate feedback on billable events, allowing the surgeon to confirm or correct the billing code determinations while the procedure is still fresh in memory. This feedback loop improves billing accuracy and reduces the need for subsequent corrections or appeals. In conventional approaches, billing code determination occurs hours or days after the procedure, when details may be forgotten or when the administrative personnel reviewing the surgical video may miss subtle but billable events.

Second, real-time billing code generation reduces the time-to-billing by eliminating the delay between procedure completion and billing code determination. This accelerates the revenue cycle and improves cash flow for healthcare providers.

Third, real-time billing code generation ensures consistency in billing code determination across surgical procedures, surgeons, and facilities by applying the same trained machine learning model to all procedures. This consistency reduces variability in billing practices and improves compliance with billing regulations.

Fourth, the automated billing report generation system 300 maintains a complete audit trail linking each billing code to the specific video segments and audio transcriptions that support the code. This documentation supports compliance audits, appeals, and defense against fraud allegations.

In contrast to the AI-assisted automated billing generation of the present invention, conventional approaches to surgical billing code generation suffer from several disadvantages. In conventional approaches, an administrative assistant, medical coder, or billing specialist must manually review the surgical video recordings and the surgical operative report after the procedure is completed. This manual review process is time-consuming, often requiring 30 minutes to several hours per procedure depending on complexity. The human reviewer may lack the medical expertise to fully understand the nuances of the surgical procedure, leading to miscoding. The time delay between the surgical procedure and the billing code determination can result in lost revenue due to missed billing opportunities. The manual review process creates bottlenecks in the revenue cycle, delaying reimbursements. Human reviewers may be inconsistent in their coding practices, leading to variability in billing across procedures and potential compliance issues.

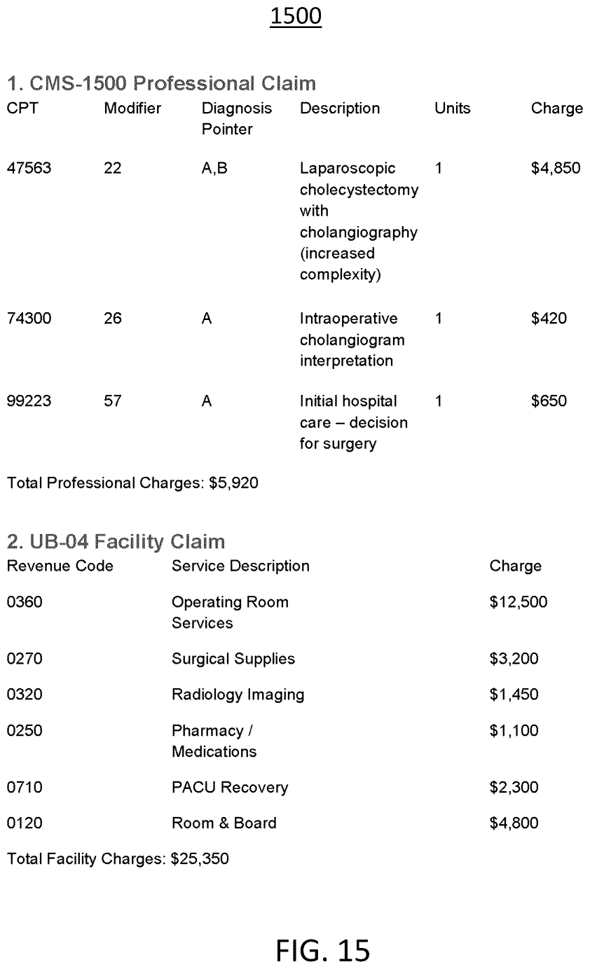

FIG. 15 shows an exemplar billing report 1500 generated by the automated billing report generation system 300, according to some embodiments. In some embodiments, the billing report 1500 may include separate sections for professional charges and facility charges. In some embodiments, the billing report 1500 may include a header section with patient identifying information (redacted for privacy), procedure date, surgeon name, and facility information. In some embodiments, the billing report 1500 may include one or more billing code summary sections listing, as non-limiting examples, one or more billing codes, any modifiers applied to the billing codes, code or service description, and charges. In some embodiments, the billing code summary may also include one or more of an associated timestamp or time range within the surgical video, a confidence score from the machine learning model, and links to the supporting video segments and audio transcriptions. In some embodiments, the billing report 1500 may also include alerts or recommendations from the billing code verification module 330 highlighting potential coding issues requiring human review.

In some embodiments, the billing report 1500 is integrated with the operative record 600, for example, as an appendix or attachment. In other embodiments, the billing report 1500 is generated as a separate document that references the operative record 600. The billing report 1500 may be exported in various formats including PDF, XML, HL7, FHIR, and other healthcare interoperability standards for integration with external systems. It should be noted that the billing report 1500 shown in FIG. 15 is merely an illustrative example and can be generated in many different formats or layouts and can include different information.

In some embodiments, the automated billing report generation system 300 further includes a billing rules engine 360 (also referred to herein as a billing rules module) configured to apply one or more billing rules in conjunction with one or more billing codes to generate a billing report and/or to determine billable amounts. In some embodiments, the billing rules engine is implemented as instructions executed by one or more processors.

In some embodiments, a billing rule includes one or more conditions and one or more outcomes, where the conditions define when a particular billing action is permitted, required, modified, reduced, increased, or suppressed, and the outcomes define the billing action to be taken. In some embodiments, the billing action includes allocating an amount, allocating a relative share of an amount, selecting between alternative codes, applying one or more modifiers, suppressing an otherwise-generated code, adding a documentation requirement to a justification record, adding a compliance flag, or routing one or more items for human review.

In some embodiments, the billing rules engine 360 is configured to receive or maintain one or more institution-specific rule sets. The institution-specific rule sets may reflect any of a facility policy, a surgeon group policy, a payer contract policy, a governmental payer policy, a specialty policy, a site-of-service policy, or a jurisdictional policy. In some embodiments, a rule set is versioned, time-stamped, and stored such that a billing report generated for a procedure is associated with the specific rule set version used at the time of generation to provide an audit trail.

In some embodiments, the billing rules define how one or more billable amounts are allocated among participants of a surgical procedure. The participants may include, for example, a primary surgeon, an assistant surgeon, a co-surgeon, a resident, a fellow, an anesthesiologist, a certified registered nurse anesthetist, a circulating nurse, a scrub nurse, a technician, and other participating clinicians or staff. In some embodiments, the billing rules engine is configured to determine participant roles using one or more of (i) scheduled staffing data, (ii) electronic health record participation logs, (iii) badge-in and badge-out logs, (iv) intraoperative device usage logs, (v) audio narration and transcribed speech indicating role and participation, and (vi) video-based recognition of role indicators and/or device interactions.

In some embodiments, the billing rules engine 360 is configured to create a participant attribution record that links a billing code, a predicted billable event, a time range in the video, and one or more participants. In some embodiments, the attribution record is used to split a professional component between multiple providers, to suppress billing for certain participants, and/or to generate participant-specific lines in the billing report 1500.

In some embodiments, the billing rules include teaching environment rules, including rules that depend on resident or fellow participation and/or supervising physician presence. In some embodiments, the system 300 generates a compliance record identifying supporting evidence of supervising physician presence during one or more portions of a procedure, where the supporting evidence includes one or more timestamps, one or more linked video segments, and one or more linked audio transcription excerpts. In some embodiments, the billing rules engine 360 is configured to require the compliance record before permitting generation of one or more billing report lines that depend on such presence.

In some embodiments, the billing rules include facility versus professional billing separation rules. In some embodiments, the billing report generation module 340 is configured to generate a facility billing portion and a professional billing portion, each portion including codes, modifiers, amounts, and evidence links corresponding to the respective portion. In some embodiments, the billing rules include time-based billing rules. The time-based billing rules may depend on a procedure duration, a billable event duration, an anesthesia time, or a documented time interval. In some embodiments, the time is computed based on one or more timestamps derived from the surgical video stream, device logs, and/or audio narration. In some embodiments, the system 300 stores the start and stop evidence for the time interval in the billing report 1500.

In some embodiments, the billing rules include bundling and unbundling rules that govern whether multiple predicted billable events should be represented by a single billing code, multiple billing codes, or a primary billing code and one or more add-on billing codes. In some embodiments, the billing code verification module 330 applies the bundling and unbundling rules to detect a discrepancy where (i) a generated code set is inconsistent with the rules, (ii) a generated code set is missing an add-on code required by the rules, or (iii) a generated code set includes a code suppressed by the rules.

In some embodiments, the billing rules engine 360 is configured to execute deterministically and efficiently by compiling a rule set into an evaluation graph and caching intermediate results across related billing lines. In some embodiments, the evaluation graph includes nodes representing conditions and outcomes, and edges representing dependencies between conditions and outcomes. In some embodiments, caching intermediate results reduces repeated evaluation of shared conditions across multiple codes and reduces end-to-end billing report generation latency.

In some embodiments, the automated billing report generation system 300 includes a modifier determination module 370, operatively coupled to the billing rules engine and configured to determine one or more modifiers to be applied to one or more billing codes and/or to one or more billing rules. In some embodiments, the modifier determination module 370 determines a modifier based on a case context that includes at least one of emergent versus scheduled status, after-hours status, trauma status, patient acuity indicators, increased complexity indicators, multiple procedure indicators, bilateral procedure indicators, altered anatomy indicators, obesity indicators, re-operation indicators, or equipment and implant usage indicators.

In some embodiments, emergent versus scheduled status is determined from one or more of (i) scheduling system data, (ii) admission or encounter metadata, (iii) surgeon dictation indicating emergent circumstances, (iv) triage metadata, and (v) video and audio evidence indicating emergent workflow patterns. In some embodiments, in response to determining that a case is emergent, the modifier determination module 370 applies a modifier to at least one billing code, applies an uplift factor to at least one computed amount, requires inclusion of a standardized justification phrase in the billing report, and/or routes the billing report for human review.

In some embodiments, the modifier determination module 370 determines one or more candidate modifiers by computing one or more procedure duration indicators from the unified procedure timeline and comparing the one or more procedure duration indicators to one or more historical baselines. In some embodiments, the historical baselines are computed from a stored corpus of prior procedures matched by at least one of procedure type, specialty, surgeon profile, facility identifier, patient acuity category, or equipment category. In some embodiments, the modifier determination module 370 computes a normalized duration deviation value representing a difference between (i) a measured duration of at least one phase of the procedure or of an identified billable event and (ii) an expected duration derived from the historical baselines. In some embodiments, in response to determining that the normalized duration deviation value exceeds a threshold and that one or more corroborating complexity indicators are present, the modifier determination module 370 recommends an increased procedural services modifier and stores, as supporting evidence, one or more timestamp ranges and one or more linked transcription excerpts corresponding to the phases or events contributing to the deviation.

In some embodiments, the modifier determination module 370 determines procedural complexity indicators from at least one of (i) features extracted from the surgical video stream, (ii) features extracted from the transcription stream, or (iii) device logs. In some embodiments, the procedural complexity indicators include at least one of increased instrument exchange frequency, increased number of discrete intervention steps, prolonged hemostasis activity, repeated dissection attempts, detected complications, dense adhesion indicators, altered anatomy indicators, obesity indicators, re-operation indicators, or implant usage indicators. In some embodiments, the modifier determination module 370 generates a complexity score and a confidence score and recommends an increased procedural services modifier when the complexity score satisfies a first predefined threshold and the confidence score satisfies a second predefined threshold, while storing a provenance record identifying the feature extraction pipeline and thresholds used to generate the recommendation.

In some embodiments, the modifier determination module 370 recommends a multiple procedure modifier in response to detecting, in the unified procedure timeline, two or more distinct billable event clusters separated by at least one of time separation, toolset separation, anatomical separation, or documentation separation. In some embodiments, time separation is determined based on non-overlapping timestamp ranges exceeding a threshold gap duration. In some embodiments, toolset separation is determined based on a change in a dominant instrument set detected in the surgical video stream and corroborated by a corresponding change in transcribed speech content. In some embodiments, documentation separation is determined based on distinct procedure step entities detected in the transcription stream that map to different procedural families or code groupings.

In some embodiments, the modifier determination module 370 recommends a distinct procedural service modifier in response to detecting a multi-site or multi-lesion intervention signal. In some embodiments, the multi-site or multi-lesion intervention signal is determined based on at least one of (i) repeated localization phrases in the transcription stream that identify different anatomical sites, (ii) video-based anatomical context classification that indicates a transition between different anatomical regions, (iii) repeated lesion treatment action sequences detected in the surgical video stream, or (iv) a combination of the above. In some embodiments, anatomical separation indicators include detected changes in anatomical landmarks, quadrant-level or segment-level region classifications, laterality indicators, or incision/port site changes. In some embodiments, in response to detecting anatomical separation indicators satisfying a predetermined threshold, the modifier determination module 370 associates the recommendation with evidence links to corresponding video segments and transcription excerpts that show the separate sites or lesions.

In some embodiments, the modifier determination module 370 recommends a co-surgeon modifier or an assistant surgeon modifier in response to determining participant participation and role attribution from at least one of scheduled staffing data, electronic health record participation logs, badge-in and badge-out logs, intraoperative device usage logs, or transcribed speech. In some embodiments, the modifier determination module 370 includes a speaker identification function that identifies, in the surgical audio stream, at least a first speaker associated with a primary surgeon and at least a second speaker associated with an assistant participant, and determines, based on speaker-identified commands, narrations, or confirmations, an assistant participation interval. In some embodiments, the assistant participation interval is corroborated by video-based recognition of role indicators comprising at least one of a second set of hands, a second instrument control pattern, assistant-specific instrument usage, or assistant-specific device interactions. In some embodiments, in response to determining that the assistant participation interval satisfies a threshold percentage of a procedure phase duration or corresponds to one or more key procedural steps, the modifier determination module 370 recommends an assistant surgeon modifier and stores evidence links supporting the participation interval.

In some embodiments, when recommending a co-surgeon modifier, the modifier determination module 370 determines that two surgeons performed distinct, contemporaneous, or alternating portions of the procedure based on at least one of (i) alternating speaker identification in the surgical audio stream, (ii) transcribed speech indicating role transitions, (iii) alternation of instrument control patterns, or (iv) separate anatomical region responsibility indicated by anatomical separation indicators. In some embodiments, the modifier determination module 370 generates a participant attribution record that assigns at least one billing line, or a portion of an amount associated with the billing line, to a co-surgeon, and stores, in association with the recommendation, one or more timestamp ranges and evidence links demonstrating the co-surgeon participation.

In some embodiments, the modifier determination module 370 selects among multiple candidate modifiers using a stored precedence table and one or more conflict rules that depend on at least one of payer identifier, institution identifier, specialty identifier, or site-of-service identifier. In some embodiments, the conflict rules specify that a distinct procedural service modifier is recommended only when anatomical separation indicators satisfy a stricter threshold than a threshold used for recommending a multiple procedure modifier. In some embodiments, the modifier determination module 370 stores, for each recommended modifier, a justification record identifying (i) the underlying trigger signals, (ii) computed scores and thresholds, and (iii) evidence links, thereby enabling the billing code verification module 330 to verify modifier selection deterministically.

In some embodiments, the automated billing report generation system 300 includes an audio processing pipeline specifically trained for an operating room environment. In some embodiments, the audio processing pipeline incorporates an operating-room-specific acoustic model trained on a multi-speaker surgical corpus that includes a plurality of roles comprising at least a primary surgeon, an assistant surgeon, an anesthesiologist, a circulating nurse, and a scrub nurse, and that further includes operating-room equipment noise and ambient speech. In some embodiments, the audio processing pipeline performs spatial preprocessing using at least one of beamforming or microphone array processing to emphasize speech arriving from an estimated direction associated with a primary speaker location and to attenuate competing speech and noise sources. In some embodiments, the audio processing pipeline further performs noise suppression and dereverberation to reduce operating-room-specific noise including suction noise, ventilator noise, instrument clatter, alarms, and electrocautery artifacts.

In some embodiments, the audio processing pipeline performs speaker diarization to segment an audio stream into speaker-homogeneous regions and assigns speaker labels to the regions. In some embodiments, the audio processing pipeline generates speaker embeddings for each region and compares the speaker embeddings to stored role-associated embeddings to perform role classification that identifies at least one of primary surgeon narration, assistant surgeon narration, anesthesia communications, nursing communications, or background staff speech. In some embodiments, the audio processing pipeline outputs a role-filtered audio stream and a role-filtered transcription stream in which at least primary surgeon narration is preferentially retained and non-target role speech is suppressed. In some embodiments, the billing code generation module 310 and the modifier determination module 370 use the role-filtered transcription stream to reduce false triggers caused by background staff communications, thereby improving precision of predicted billable event detection and reducing spurious modifier recommendations.

In some embodiments, the audio processing pipeline outputs, for each transcription token or phrase, a role label and a role confidence score, and the billing code machine learning model 350 uses the role label and role confidence score as additional features when predicting billing codes. In some embodiments, in response to determining that a transcription excerpt has a role label inconsistent with a billing-event narration role requirement, the system suppresses the excerpt for billing-event inference or routes the corresponding predicted event for review. In some embodiments, isolating role-specific speech reduces false event triggers in downstream billing inference by preventing incidental utterances by non-surgeon staff from satisfying billing-event keyword conditions or rule-required phrase conditions. In some embodiments, isolating role-specific speech improves temporal precision by reducing transcription instability due to overlapping speakers and by improving alignment between event timestamps and corresponding narration excerpts.

In some embodiments, the billing rules engine 360 is configured to apply a hierarchy of modifiers. In some embodiments, at least a first modifier changes an amount determination associated with a billing code, at least a second modifier changes an eligibility rule associated with the billing code, and at least a third modifier changes a documentation requirement associated with the billing code. In some embodiments, conflicts between modifiers are resolved using a stored precedence table associated with a payer and/or an institution.

In some embodiments, the billing code database 320 stores, for each billing code, one or more rule hooks that identify billing rules that commonly apply to the code. In some embodiments, the billing rules engine 360 uses the rule hooks to automatically select a subset of billing rules to evaluate for a predicted code set, thereby reducing compute time and improving determinism. In some embodiments, the automated billing report generation system 300 is further configured to perform intelligent billing edits by evaluating candidate billing codes against stored code-pair edit rules. In some embodiments, the code-pair edit rules include at least one national edit table and at least one payer-specific edit table, where the edit tables define, for pairs or groups of codes, one or more outcomes comprising mutually inclusive determinations, mutually exclusive determinations, conditional-allowability determinations dependent on modifiers, and documentation or evidence requirements.

In some embodiments, the billing code verification module 330 and/or the billing rules engine 360 applies the code-pair edit rules to a candidate code set generated by the billing code generation module 310 to automatically suppress one or more mutually inclusive codes that are not permitted to be billed together under an applicable edit table. In some embodiments, suppression comprises removing a suppressed code from a finalized code set while retaining, in an audit trail, an identifier of the suppressed code, an identifier of an edit rule that caused suppression, and one or more evidence links corresponding to the underlying detected event that produced the suppressed code. In some embodiments, the system evaluates whether an unbundling recommendation is permitted when supported by multimodal evidence. In some embodiments, in response to determining that (i) a code pair is typically treated as bundled under an edit table and (ii) an unbundling condition is satisfied based on the detected multimodal evidence, the system 300 generates an unbundling recommendation that includes a recommended modifier, a justification record, and evidence links to at least one time-indexed media segment and at least one transcription excerpt supporting the unbundling condition. In some embodiments, the multimodal evidence includes at least one of (i) video-based detection of distinct intervention steps separated in time or anatomy, (ii) transcription-based detection of separate procedural intent, or (iii) device-log corroboration of separate instrumentation or implants.

In some embodiments, in response to determining that a candidate code set includes a mutually exclusive code combination under an applicable edit table, the system 300 generates a compliance alert prior to claim submission and routes the affected billing lines for review. In some embodiments, the compliance alert includes an edit-table identifier, a rule identifier, a description of the conflicting code combination, and evidence links to the time ranges and transcription excerpts that caused generation of the mutually exclusive codes. In some embodiments, the system 300 applies payer-specific adjudication profiles that modify one or more of code selection, modifier application, bundling or unbundling outcomes, or reimbursement prediction based on an identified payer. In some embodiments, the identified payer is determined from scheduling metadata, encounter metadata, eligibility verification records, or claim destination identifiers. In some embodiments, an adjudication profile defines payer-specific overrides including at least one of (i) edit-table selection precedence, (ii) modifier precedence rules, (iii) coverage or eligibility constraints, (iv) documentation or evidence sufficiency requirements, or (v) payment policy adjustments used to compute a predicted reimbursement amount.

In some embodiments, the system 300 maintains a continuously updated rules database storing rule sets and edit tables reflecting governmental payer policies and commercial payer policies. In some embodiments, updates to the rules database include one or more of new edit rules, modified edit rules, deprecated edit rules, new payer profiles, modified payer profiles, or revised evidence requirements. In some embodiments, each update is versioned and time-stamped, and the billing report 1500 stores identifiers of the versions used to generate the billing lines to support repeatability and auditability. In some embodiments, each generated billing code, each billing line, and each compliance alert is automatically associated with a corresponding time-indexed evidentiary media segment derived from a unified procedure timeline index referenced to a monotonic time base. In some embodiments, the system generates an auditable linkage record that maps a billing line identifier to (i) a timestamp range, (ii) a video segment identifier when available, (iii) an audio segment identifier when available, and (iv) a transcription excerpt identifier when available, where the linkage record is stored in association with the billing report 1500.

In some embodiments, the narrative generation module 390, described later, uses the auditable linkage record to generate justification text that includes evidence anchors derived from the time-indexed evidentiary media segments. In some embodiments, the narrative generation module 390 inserts, for a billing line, at least one evidence-anchored citation comprising a timestamp range and a reference to at least one of a video segment identifier, an audio segment identifier, or a transcription excerpt identifier, thereby enabling downstream compliance review, audit defense, and clinical validation without re-locating evidence in the underlying surgical media streams.

In some embodiments, the billing report generation module 340 is configured to generate a billing report 1500 that includes, for each billing line, (i) a billing code, (ii) one or more applied modifiers, (iii) a participant attribution, (iv) a time range or timestamp, (v) a confidence score, (vi) an amount or relative allocation, (vii) one or more evidence links to supporting video segments and audio transcriptions, and (viii) a justification text field configured to include a narrative description supporting the billing line.

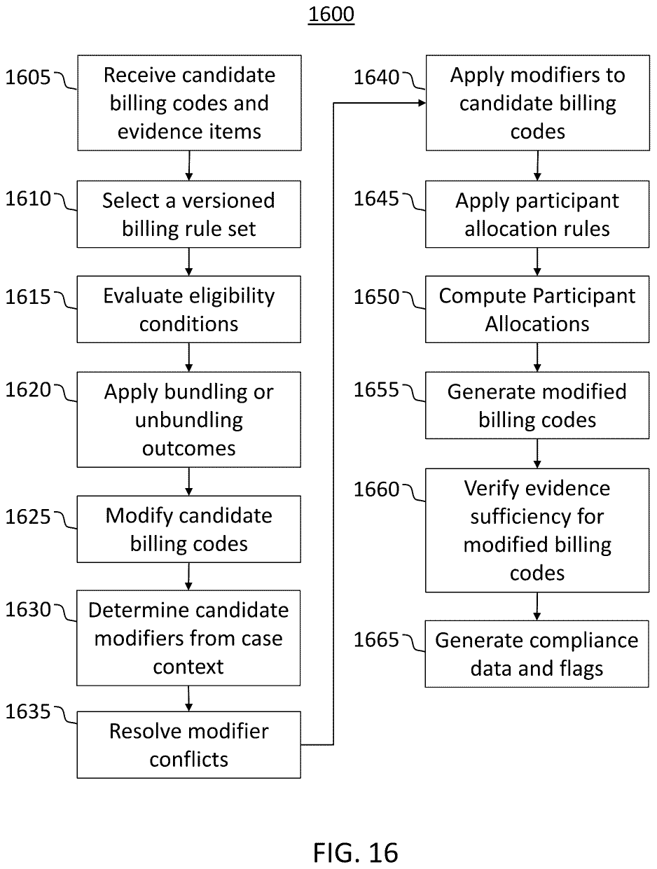

FIG. 16 illustrates a flowchart of an exemplar method 1600 for billing code processing and billing-line computation performed by the automated billing report generation system 300 using the billing rules engine 360 and the modifier determination module 370, according to some embodiments. In some embodiments, the method 1600 begins at step 1605 in which the system 300 receives a set of candidate billing codes for a surgical procedure together with associated evidence items. In some embodiments, an evidence item comprises at least one of a timestamp range, a video segment identifier, an audio segment identifier, or a transcription excerpt identifier, for example as derived from a unified procedure timeline index.

In step 1610, the system 300 selects a versioned billing rule set applicable to at least a subset of the candidate billing codes. In some embodiments, selecting the versioned billing rule set is based on at least one of an institution identifier, a payer identifier, a specialty identifier, or a site-of-service identifier. In some embodiments, the selected versioned billing rule set is stored with the billing report 1500 to provide repeatable computation and an audit trail. In step 1615, the billing rules engine 360 evaluates one or more eligibility conditions of the selected rule set. In some embodiments, evaluating eligibility conditions comprises determining whether at least one candidate billing code is permitted in view of one or more rule conditions including documentation sufficiency, participant requirements, site-of-service requirements, or time-based requirements. In step 1620, the billing rules engine 360 applies one or more bundling or unbundling outcomes of the selected rule set. In some embodiments, bundling or unbundling outcomes cause the billing rules engine 360 to suppress at least one candidate billing code, add at least one additional billing code, and/or replace a subset of the candidate billing codes with a different set of billing codes, while retaining one or more evidence items for auditability.

In step 1625, the system 300 modifies the candidate billing codes based on the evaluated eligibility conditions and the applied bundling or unbundling outcomes to generate an intermediate code set. In some embodiments, the intermediate code set includes one or more codes tagged as suppressed-by-rules and one or more codes tagged as added-by-rules, each tag being stored with an identifier of a rule outcome that caused the modification.

In step 1630, the modifier determination module 370 determines one or more candidate modifiers from a case context derived from at least one of scheduling metadata, encounter metadata, the transcription stream, the surgical video stream, or participant involvement data. In some embodiments, the candidate modifiers include at least one of an emergent-versus-scheduled modifier, a multiple procedure modifier, an increased procedural services modifier, a distinct procedural service modifier, an assistant surgeon modifier, or a co-surgeon modifier. In step 1635, the system 300 resolves modifier conflicts. In some embodiments, resolving modifier conflicts comprises selecting among multiple candidate modifiers using a stored modifier precedence table associated with at least one of a payer identifier or an institution identifier and, for example, suppressing at least one conflicting modifier while retaining, in an audit trail, an identifier of the suppressed modifier and an identifier of a conflict rule that caused suppression. In step 1640, the system 300 applies the resolved modifiers to the intermediate code set to generate a modifier-applied code set. In some embodiments, applying the resolved modifiers comprises updating at least one amount computation rule, eligibility rule, documentation requirement, or bundling edit condition associated with at least one code in the modifier-applied code set.

In step 1645, the billing rules engine 360 applies participant allocation rules to determine how at least one amount is allocated among a plurality of participants in the surgical procedure. In some embodiments, the participants include at least one of a primary surgeon, an assistant surgeon, a co-surgeon, a resident, a fellow, an anesthesiologist, a certified registered nurse anesthetist, a circulating nurse, a scrub nurse, or a technician. In some embodiments, applying participant allocation rules comprises generating a participant attribution record linking at least one billing line to at least one participant and to at least one evidence item. In step 1650, the system 300 computes participant allocations. In some embodiments, computing participant allocations comprises computing, for at least one billing line, at least one of an allocated amount, an allocated percentage, or an allocated RVU value for each of one or more participants, based on the modifier-applied code set and at least one rule outcome of the selected versioned billing rule set.

In step 1655, the system 300 generates modified billing codes and corresponding billing lines. In some embodiments, each billing line includes a billing code, one or more applied modifiers, an amount or an allocation of an amount, a confidence score, and at least one evidence link to a corresponding time range or segment identifier derived from the unified procedure timeline. In step 1660, the system 300 verifies evidence sufficiency for the modified billing codes. In some embodiments, verifying evidence sufficiency comprises determining whether a billing line satisfies one or more rule-set-specific evidence requirements including a minimum-duration video segment requirement, a required phrase requirement in a transcription excerpt, a participant presence requirement, and/or device-log corroboration. In step 1665, the system 300 generates compliance data and flags. In some embodiments, the compliance data includes one or more of an auditable linkage record mapping a billing line identifier to evidence items, a rule set version identifier, a phrase library version identifier, a modifier determination logic version identifier, and one or more compliance alerts identifying missing or inconsistent evidence requirements, thereby supporting downstream compliance review and audit.

In some embodiments, the automated billing report generation system 300 further includes a relative value unit optimization engine (RVU optimization engine) 380 configured to evaluate predicted billing codes and corresponding billing lines relative to stored relative value unit (RVU) assignments and to generate one or more machine-generated outputs comprising (i) an RVU consistency status, (ii) a documentation opportunity record, (iii) a code review alert, (iv) a recommended modifier, (v) a recommended add-on code, or (vi) a recommended documentation prompt anchored to one or more evidence items. In some embodiments, an RVU is a numeric value associated with a billing code and configured to represent, in a normalized manner, an expected relative resource representation for performing a corresponding service. In some embodiments, the RVU associated with a billing code includes one or more RVU components including a work RVU component, a practice expense RVU component, and a malpractice RVU component. In some embodiments, the billing code database 320 stores, for each billing code, at least one RVU value, an RVU component breakdown, and one or more applicability constraints comprising a site-of-service constraint, a specialty constraint, a payer constraint, and an effective date range.

In some embodiments, the RVU optimization engine 380 is configured to select, from the billing code database 320, an RVU table version based on at least one of an institution identifier, a payer identifier, a jurisdiction identifier, a specialty identifier, or a site-of-service identifier. In some embodiments, the RVU optimization engine 380 stores an identifier of the RVU table version in association with the billing report 1500 to support repeatable evaluation and auditability of RVU-based determinations.

In some embodiments, the RVU optimization engine 380 computes a procedural complexity score indicative of procedural complexity for the surgical procedure. In some embodiments, the procedural complexity score is computed using one or more multimodal features comprising at least one of (i) operative time computed from timestamps in the unified procedure timeline, (ii) a number of discrete intervention steps detected from at least one of video-based action recognition events or transcription-derived procedural step entities, (iii) instrument exchange patterns determined from video-based instrument identification and temporal transitions, (iv) detected complications determined from at least one of a complication classifier applied to video segments or a complication entity detected in transcribed speech, (v) tissue condition indicators determined from video-based image features indicative of bleeding, edema, adhesions, or friability, or (vi) anatomical difficulty markers determined from anatomical context classifiers applied to video frames. In some embodiments, the RVU optimization engine 380 computes the procedural complexity score using a weighted scoring function that is executed deterministically on extracted features and produces a score having an associated confidence value. In some embodiments, the RVU optimization engine 380 additionally computes one or more intermediate sub-scores comprising a time sub-score, a step-count sub-score, an instrument-exchange sub-score, and a complication sub-score, and caches the sub-scores for reuse when generating multiple billing lines for the same procedure.

In some embodiments, the RVU optimization engine 380 maps the procedural complexity score to one or more candidate code sets by selecting, from a stored mapping, one or more billing codes having RVU values within a target RVU range associated with the procedural complexity score and a procedure context. In some embodiments, the procedure context includes at least one of a detected procedure family, an anatomical region identifier, a specialty identifier, or a site-of-service identifier.

In some embodiments, the RVU optimization engine 380 performs undercoding detection by comparing an RVU value associated with at least one predicted billing code to an expected RVU range associated with the procedural complexity score. In some embodiments, in response to determining that the RVU value for the predicted billing code is below a lower bound of the expected RVU range by more than a threshold, the RVU optimization engine 380 generates an undercoding alert and associates the undercoding alert with one or more evidence items comprising at least one of a video segment identifier, an audio segment identifier, or a transcription excerpt identifier. In some embodiments, the RVU optimization engine 380 performs overcoding risk detection by comparing an RVU value associated with at least one predicted billing code to an expected RVU range associated with the procedural complexity score. In some embodiments, in response to determining that the RVU value for the predicted billing code exceeds an upper bound of the expected RVU range by more than a threshold, the RVU optimization engine 380 generates an overcoding risk flag and routes the billing line for review while retaining an evidence link to one or more supporting media segments.

In some embodiments, the RVU optimization engine 380 performs missed charge component detection by analyzing the operative record 600 and the billing report 1500, in combination with all of the multimodal data, to detect performed but not yet represented billable components. In some embodiments, the performed but not yet represented billable components are detected based on at least one of (i) recognized surgical actions associated with add-on codes, (ii) detected use of specialized equipment or implants from video-based object detection and/or device logs, (iii) transcription excerpts indicating performance of a billable component, or (iv) temporal patterns indicative of prolonged or repeated interventions.

In some embodiments, in response to detecting a performed but not yet represented billable component, the RVU optimization engine 380 generates a suggested add-on code record that includes (i) a candidate add-on billing code, (ii) a confidence score, (iii) a timestamp range, and (iv) one or more evidence links anchored to the synchronized multimodal record. In some embodiments, the suggested add-on code record is provided to the billing rules engine 360 such that bundling, unbundling, and eligibility conditions are evaluated before the suggested add-on code is included in a finalized billing report.

In some embodiments, the RVU optimization engine 380 generates documentation opportunity records in response to detecting that a predicted billing code, a modifier, or an add-on code is permitted by an applicable rule set but requires additional documentation or evidence anchors. In some embodiments, the documentation opportunity record identifies (i) a target billing line, (ii) a missing documentation element, (iii) a recommended standardized phrase from a phrase library, and (iv) at least one candidate evidence segment to link.

In some embodiments, the RVU optimization engine 380 generates near-real-time prompts in response to detecting, during or shortly after the procedure, that a documentation opportunity record has been created. In some embodiments, a prompt is configured for minimal interaction and is presentable via at least one of a surgical console interface, a heads-up display, a mobile device, or a post-procedure review dashboard. In some embodiments, the prompt includes a short confirmation query and a selectable option set configured to be answered via a short spoken response, thereby reducing interaction events while preserving evidence linkage. In some embodiments, the RVU optimization engine 380 applies a prompt suppression policy configured to reduce distraction and reduce compute load by limiting prompt generation to conditions satisfying at least one threshold comprising an RVU deviation threshold, a confidence threshold, a required-evidence threshold, or a procedure phase threshold. In some embodiments, the prompt suppression policy is executed using a deterministic rule evaluation that is cached per procedure phase.

In some embodiments, the RVU optimization engine 380 operates in conjunction with the modifier determination module 370 to recommend one or more modifiers that affect RVU-relevant computation and/or amount computation. In some embodiments, the RVU optimization engine 380 recommends an emergent-versus-scheduled modifier in response to a detected emergent workflow indicator and generates an evidence-anchored justification record identifying one or more transcription excerpts and one or more timestamp ranges supporting the emergent workflow indicator.

In some embodiments, the RVU optimization engine 380 operates in conjunction with participant allocation rules executed by the billing rules engine 360 to compute participant-specific RVU allocations. In some embodiments, the RVU optimization engine 380 outputs, for at least one billing line, a participant RVU allocation record that maps at least one participant identifier to at least one of an allocated RVU amount, an allocated percentage, or an allocated billing amount computed from allocated RVUs and a stored conversion parameter. In some embodiments, the RVU optimization engine 380 maintains surgeon-specific documentation profiles derived from historical procedures and post-procedure correction data. In some embodiments, the surgeon-specific documentation profile includes one or more recurring documentation omission patterns detected by comparing (i) initial predicted billing codes and documentation opportunity records to (ii) finalized billing codes after review. In some embodiments, the RVU optimization engine 380 uses the surgeon-specific documentation profile to adjust one or more thresholds for undercoding detection, missed charge component detection, or prompt suppression on a per-user basis while maintaining versioned configuration identifiers for reproducibility.

In some embodiments, the RVU optimization engine 380 is configured to reduce computational overhead by incrementally updating the procedural complexity score and associated expected RVU range as new evidence items are appended to a real-time event ledger. In some embodiments, incremental updating comprises recomputing only a subset of feature-derived sub-scores affected by newly received events and reusing cached sub-scores unaffected by the newly received events.

In some embodiments, the RVU optimization engine 380 generates, stores, and exports an RVU evaluation record that includes (i) an identifier of the RVU table version, (ii) the procedural complexity score and any sub-scores, (iii) an expected RVU range, (iv) one or more RVU deviation determinations, and (v) evidence links supporting the determinations. In some embodiments, the RVU evaluation record is stored in association with the billing report 1500 and is retrievable for repeatable evaluation in response to later rule updates or payer-specific configuration changes.

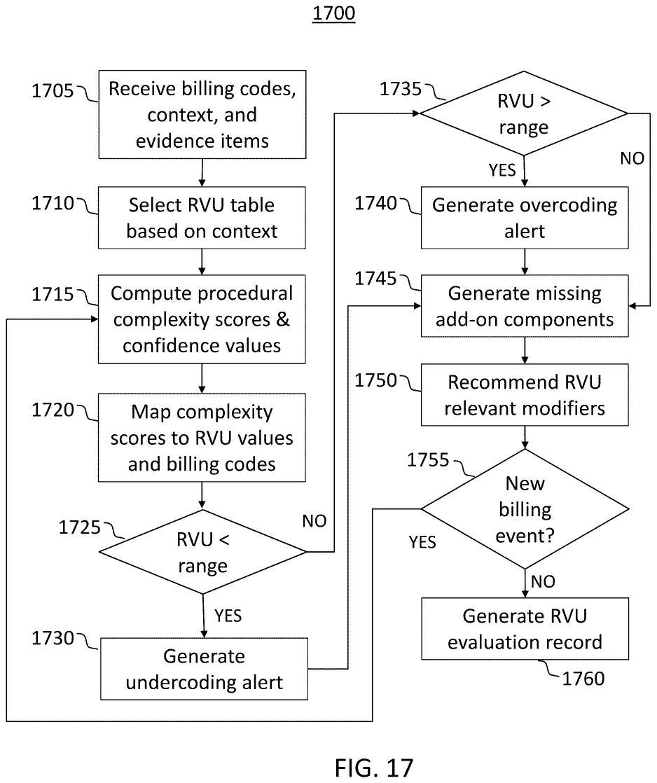

FIG. 17 illustrates a flowchart of an exemplar method 1700 for RVU-based evaluation and recommendation performed by the RVU optimization engine 380, according to some embodiments. In some embodiments, method 1700 is executed using time-indexed evidence items derived from the unified procedure timeline and in coordination with at least one of the billing code generation module 310, the billing rules engine 360, the modifier determination module 370, and the billing report generation module 340.

In step 1705, the RVU optimization engine 380 receives one or more billing codes for a surgical procedure together with a case context and one or more evidence items. In some embodiments, the case context includes at least one of a procedure family identifier, a specialty identifier, a site-of-service identifier, a payer identifier, an institution identifier, or a participant attribution context. In some embodiments, the evidence items include one or more of timestamp ranges, video segment identifiers, audio segment identifiers, transcription excerpt identifiers, or device log entries. In step 1710, the RVU optimization engine 380 selects an RVU table based on the case context. In some embodiments, selecting the RVU table includes selecting a versioned RVU table based on at least one of the payer identifier, institution identifier, jurisdiction identifier, specialty identifier, or site-of-service identifier, and storing an RVU table version identifier in association with an RVU evaluation record.

In step 1715, the RVU optimization engine 380 computes one or more procedural complexity scores and corresponding confidence values. In some embodiments, the procedural complexity scores are computed using multimodal features that include at least one of operative time, a number of discrete intervention steps, instrument exchange patterns, detected complications, tissue condition indicators, or anatomical difficulty markers derived from at least one of the surgical video stream, the surgical audio stream, the transcription stream, or device logs. In step 1720, the RVU optimization engine 380 maps the one or more procedural complexity scores to one or more RVU values and candidate billing code ranges. In some embodiments, mapping comprises selecting a target RVU range corresponding to a procedural complexity score and identifying one or more candidate billing codes having RVU values within the target RVU range for a corresponding procedure context. In step 1725, the RVU optimization engine 380 determines whether an RVU value associated with at least one received or predicted billing code is below an expected RVU range.

In some embodiments, in response to determining that the RVU value is below the expected RVU range, the method proceeds to step 1730. In step 1730, the RVU optimization engine 380 generates an undercoding alert. In some embodiments, the undercoding alert includes a recommended code adjustment, a recommended add-on code candidate, a recommended modifier candidate, and one or more evidence links supporting the recommendation. After generating the undercoding alert, the method continues with step 1745.

If, in step 1725, the RVU optimization engine 380 determines the RVU value associated with at least one received or predicted billing code is not below an expected RVU range, then, the method proceeds to step 1735. In step 1735, the RVU optimization engine 380 determines whether the RVU value associated with at least one received or predicted billing code is above an expected RVU range. In some embodiments, in response to determining that the RVU value is above the expected RVU range, the method proceeds to step 1740. In step 1740, the RVU optimization engine 380 generates an overcoding alert. In some embodiments, the overcoding alert includes a compliance flag, a recommendation to route a billing line for review, and one or more evidence links supporting the overcoding risk determination.

In step 1745, the RVU optimization engine 380 generates one or more missing add-on component determinations. In some embodiments, generating missing add-on component determinations comprises analyzing the time-indexed evidence items to detect performed but not yet represented billable components and generating, for each detected component, a suggested add-on code record including a confidence score and one or more evidence links. In step 1750, the RVU optimization engine 380 generates recommendations for one or more RVU-relevant modifiers. In some embodiments, recommending RVU-relevant modifiers comprises determining one or more candidate modifiers based on at least one of an emergent workflow indicator, a normalized duration deviation value relative to historical baselines, a procedural complexity score, an anatomical separation indicator, or a participant involvement indicator, and generating a justification record with evidence links for each recommended modifier.

In step 1755, the system 300 monitors a real time event log for the surgical procedure and determines whether a new billing event has been detected. In some embodiments, a new billing event includes at least one of a newly detected billable component, a newly corroborated predicted event, or a rule-driven addition or suppression of a billing code. In some embodiments, in response to determining that a new billing event has been detected, the RVU optimization engine 380 returns to step 1705 to perform RVU evaluation on an updated set of billing codes and evidence items. In step 1760, the RVU optimization engine 380 generates an RVU evaluation record. In some embodiments, the RVU evaluation record includes an RVU table version identifier, a procedural complexity score and confidence value, an expected RVU range, one or more RVU deviation determinations, one or more alerts or recommendations, and one or more evidence links supporting the determinations, and is stored in association with the billing report 1500 to support repeatable review and audit.

In some embodiments, the automated billing report generation system 300 further includes a narrative generation module 390 configured to generate a justification text field for at least one billing line. In some embodiments, the narrative generation module 390 includes a large language model (LLM) configured to generate natural language text in response to one or more inputs including the predicted billing codes, applied modifiers, participant attribution records, extracted evidence, and predetermined standardized phrases.

In some embodiments, the narrative generation module 390 is configured to generate standardized phrases that may be required by an institution, payer, or compliance policy, and to insert such standardized phrases into the justification text field. In some embodiments, the standardized phrases are stored in a phrase library associated with a template selected for a surgical specialty, a payer, and/or an institution. In some embodiments, the phrase library is versioned and the billing report 1500 stores an identifier of the phrase library version used for report generation.

In some embodiments, the narrative generation module 390 is configured to generate one or more code-specific descriptions describing why a code applies, based on evidence extracted from the surgical video stream and associated audio narration. In some embodiments, the narrative generation module 390 is configured to include citations in the form of evidence links, timestamps, and/or quoted transcription excerpts, such that a human reviewer can rapidly confirm that the justification is supported by the operative record 600 and associated multimedia.

In some embodiments, the narrative generation module 390 is configured to generate different narrative styles for different output targets. The output targets may include a claim submission narrative, an internal coding note, an appeal letter draft, a compliance audit note, or a patient-facing explanation. In some embodiments, the style is selected based on an export format selection including at least one of PDF, XML, HL7, or FHIR, and/or based on a destination system identifier.

In some embodiments, the narrative generation module 390 is configured to operate under one or more constraints that reduce unsupported text generation. In some embodiments, the constraints include requiring that each material factual statement in the generated narrative be anchored to a stored evidence item including at least one of a video segment identifier, a timestamp, an audio segment identifier, a transcription excerpt, or an operative report section reference. In some embodiments, when sufficient evidence is not available to support a statement, the narrative generation module 390 inserts a prompt for human input and/or routes the line for human review.

In some embodiments, the narrative generation module 390 is configured to generate multiple candidate narratives and select a candidate based on one or more scoring criteria including completeness, presence of required standardized phrases, presence of evidence anchors, and consistency with applied billing rules. In some embodiments, the selected candidate narrative and the scoring criteria results are stored as part of the billing report 1500 audit trail.

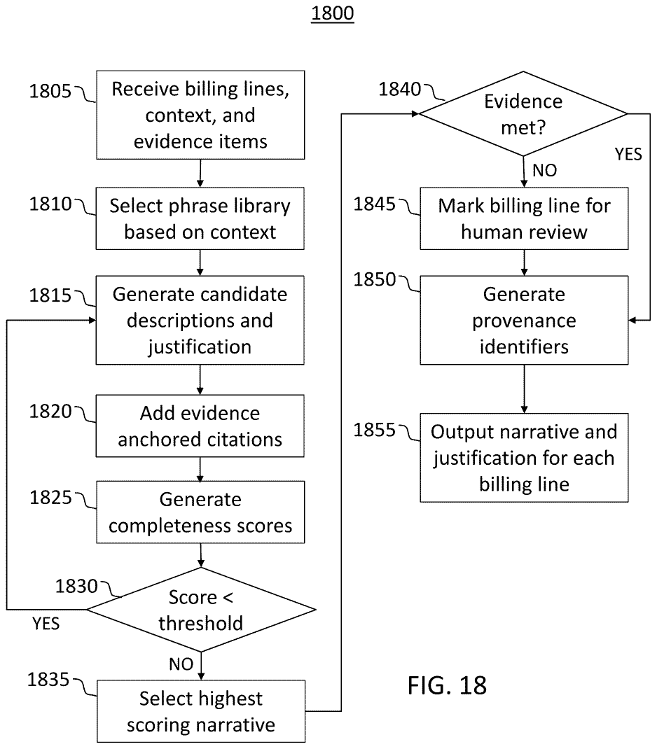

FIG. 18 illustrates a flowchart of an exemplar method 1800 for generating evidence-anchored descriptions and justifications for billing lines in the billing report 1500, according to some embodiments. In some embodiments, method 1800 is performed by the narrative generation module 390 in conjunction with at least one of the billing rules engine 360, the billing code verification module 330, and a time-indexed evidence structure derived from the unified monotonic procedure index. In step 1805, the narrative generation module 390 receives a plurality of billing lines in the billing report 1500 together with a context and one or more associated evidence items. In some embodiments, the context includes at least one of a payer identifier, an institution identifier, a specialty identifier, a site-of-service identifier, a procedure family identifier, a selected export format, or a destination system identifier. In some embodiments, an evidence item comprises at least one of a timestamp range, a video segment identifier, an audio segment identifier, or a transcription excerpt identifier corresponding to the billing line.

In step 1810, the narrative generation module 390 selects a phrase library based on the context. In some embodiments, the phrase library includes standardized phrases required by at least one of an institution policy, a payer policy, or a compliance policy. In some embodiments, the phrase library is versioned and the system stores an identifier of the selected phrase library version in association with the billing report 1500 to support repeatability. In step 1815, the narrative generation module 390 generates one or more candidate narrative descriptions and justifications for at least one billing line. In some embodiments, generating the candidate narratives comprises conditioning a large language model on (i) the billing code, (ii) one or more applied modifiers, (iii) a participant attribution record when applicable, (iv) the selected standardized phrases, and (v) one or more evidence items linked to the billing line. In step 1820, the narrative generation module 390 adds evidence-anchored citations to the candidate narrative descriptions and justifications. In some embodiments, an evidence-anchored citation includes at least one of a timestamp range, a reference to a video segment identifier, a reference to an audio segment identifier, or a quoted transcription excerpt identifier. In some embodiments, adding evidence-anchored citations comprises selecting, from an auditable linkage record, one or more evidence items that satisfy an evidence requirement associated with the billing line.

In step 1825, the narrative generation module 390 generates a completeness score for each candidate narrative. In some embodiments, the completeness score is computed based on satisfaction of one or more constraints including presence of required standardized phrases, presence of at least one evidence-anchored citation per sentence, consistency with applied billing rules, and absence of unsupported clinical assertions. In some embodiments, the completeness score is computed deterministically using a weighted scoring function applied to extracted narrative features. In step 1830, the narrative generation module 390 determines whether a completeness condition is met based on the completeness scores. In some embodiments, the completeness condition is configurable to be satisfied when at least one, some, a majority, or all completeness scores for the candidate narratives are at or above a threshold. In some embodiments, the threshold and a selection of how the completeness condition is met are user-configurable based on at least one of an institution policy, a payer policy, a specialty policy, or a destination system requirement. In some embodiments, responsive to determining that the completeness condition is not met, the system returns to step 1815 to generate one or more additional candidate narratives or modify one or more of the candidate narratives, and/or to step 1820 to insert additional evidence-anchored citations.

In step 1835, the narrative generation module 390 selects the highest scoring narrative from among the candidate narratives for each billing line in the billing report 1500. In some embodiments, selecting the highest scoring narrative comprises selecting a narrative that satisfies required-phrase constraints and evidence-anchoring constraints while having a maximum completeness score. In step 1840, the system evaluates whether an evidence sufficiency condition is met for the selected narrative and the corresponding billing line. In some embodiments, the evidence sufficiency condition comprises at least one of a minimum-duration linked video segment requirement, a required-phrase presence requirement in a transcription excerpt, a participant presence requirement, or a corroborating device-log requirement. In step 1845, in response to determining that the evidence sufficiency condition is not met, the system marks the billing line for human review. In some embodiments, marking the billing line for human review includes storing an identification of a missing evidence item and storing a recommended corrective action comprising capturing additional evidence, linking an additional evidence segment, or adding a standardized phrase.

In step 1850, the narrative generation module 390 generates provenance identifiers for the selected narrative. In some embodiments, the provenance identifiers include at least one of a large language model configuration identifier, a prompt template identifier, a phrase library version identifier, a billing rules engine version identifier, a billing rule set version identifier, or an auditable linkage record identifier, and the system stores the provenance identifiers in association with the billing report 1500. In step 1855, the system 300 outputs, for each billing line, the selected narrative description and justification together with one or more evidence-anchored citations and any associated provenance identifiers. In some embodiments, the narrative description and justification are stored in a justification text field in the billing report 1500 and are exportable with the billing report in at least one of a machine-readable representation or a human-readable rendering.

In some embodiments, the billing code verification module 330 is configured to verify not only codes but also rule application. In some embodiments, the verification includes determining whether (i) a required modifier was applied, (ii) a prohibited modifier was applied, (iii) a required participant attribution is missing, (iv) a required standardized phrase is missing, or (v) a computed amount is inconsistent with an applied rule set. In some embodiments, in response to a failed verification, the system 300 generates an alert, generates a recommended correction, and stores the alert and recommended correction in association with the billing report 1500.

In some embodiments, the billing code verification module 330 includes an evidence sufficiency analyzer configured to determine whether a billing line satisfies evidence requirements. In some embodiments, the evidence requirements are rule-set specific and include one or more of presence of a minimum-duration video segment, presence of a supporting transcription excerpt containing a required phrase, presence of a supervising physician presence indicator, and presence of device log corroboration. In some embodiments, in response to determining that evidence is insufficient, the system 300 marks the billing line as requiring review and stores a description of missing evidence items.

In some embodiments, the billing rules engine 360 is configured to support multiple billing procedures for different organizations by allowing configuration of one or more of (i) participant role definitions, (ii) supervision requirements, (iii) allocation formulas, (iv) modifier precedence, (v) required phrase libraries, (vi) evidence requirements, (vii) review thresholds based on confidence scores, and (viii) export mappings to external systems. In some embodiments, the configuration is performed through an administrative user interface requiring authenticated access and the configuration changes are logged.

In some embodiments, the system 300 is configured to perform reconciliation between (i) predicted billing codes generated in real-time during the procedure and (ii) billing codes generated after the procedure based on the finalized operative record 600. In some embodiments, the reconciliation identifies differences, determines whether differences are explained by post-procedure edits, rule updates, or evidence updates, and generates a reconciliation section in the billing report 1500.

In some embodiments, the system 300 is configured to generate a denial risk indicator for at least one billing line and/or for a proposed claim package prior to claim submission using a denial risk model trained on historical claim adjudication data. In some embodiments, the historical claim adjudication data includes, for a plurality of past claims, at least one of submitted billing codes, applied modifiers, diagnosis codes, payer identifiers, site-of-service identifiers, submitted documentation artifacts, evidence sufficiency determinations, claim outcomes, denial reason codes, and appeal outcomes.

In some embodiments, the denial risk model generates, for each proposed claim or billing line, a denial risk score computed from one or more input signals comprising documentation completeness, diagnosis-procedure congruence, modifier usage patterns, payer-specific historical behavior, and detected compliance anomalies. In some embodiments, documentation completeness is determined based on satisfaction of a template-specific checklist and one or more evidence sufficiency requirements; diagnosis-procedure congruence is determined by comparing one or more diagnosis entities extracted from the operative record 600 and/or the transcription stream to one or more procedure-family entities associated with the proposed billing codes; modifier usage patterns include one or more of modifier frequency, modifier combinations, modifier ordering, or modifier-payer interaction indicators; payer-specific historical behavior includes a payer-specific denial baseline and payer-specific denial reason distributions; and detected compliance anomalies include one or more of mutually exclusive code pair indicators, bundling edit conflicts, missing required phrases, or missing supervising physician presence evidence.

In some embodiments, the denial risk model comprises a machine learning model configured to output the denial risk score and at least one explanatory output comprising a ranked list of contributing factors, a denial reason prediction, or an evidence insufficiency indicator. In some embodiments, the system stores, in association with the denial risk score, a model version identifier and one or more feature identifiers to support repeatability and auditability. In some embodiments, responsive to determining that the denial risk score exceeds a configurable threshold, the system 300 generates at least one of (i) a corrective documentation prompt, (ii) a recommended modifier adjustment, (iii) a recommended code adjustment, or (iv) a claim review alert prior to claim submission. In some embodiments, the corrective documentation prompt is configured for minimal interaction and includes a short standardized phrase suggestion from a phrase library and an evidence link to a corresponding time-indexed media segment to facilitate rapid correction. In some embodiments, the configurable threshold is selected based on at least one of payer identifier, institution identifier, specialty identifier, site-of-service identifier, or procedure family, and is versioned such that the billing report 1500 stores an identifier of the threshold configuration used. In some embodiments, the system 300 routes billing lines exceeding the configurable threshold to a review queue and generates a denial-risk mitigation record that identifies (i) the triggering factor, (ii) a recommended corrective action, and (iii) one or more evidence links supporting the recommended corrective action.

In some embodiments, the billing rules engine 360 and narrative generation module 390 cooperate such that, when a billing rule requires a particular justification, the narrative generation module 390 generates a rule-compliant narrative including required standardized phrases and evidence anchors corresponding to the rule. In some embodiments, when the modifier determination module 370 applies an emergent modifier, the narrative generation module generates text describing emergent circumstances and includes evidence anchors corresponding to the determination.

In some embodiments, the operative report generation system 200 and the automated billing report generation system 300 may be extended with additional AI-assisted functionality to further improve surgical report generation, billing generation, and validation/verification processes. In some embodiments, the automated billing report generation system 300 includes a predictive coding module (not illustrated) that analyzes the pre-operative diagnosis, planned procedure, and patient history to predict likely billing codes before the surgery begins. During the procedure, the predicted codes are compared against the actual billable events identified by the billing code machine learning model 350. Discrepancies between predicted and actual codes are flagged for review, potentially identifying documentation gaps or coding opportunities.

In some embodiments, the automated billing report generation system 300 includes a compliance monitoring module (not illustrated) that continuously monitors billing code patterns across procedures, surgeons, and facilities to identify potential compliance risks such as upcoding, unbundling, or systematic undercoding. The compliance monitoring module generates periodic reports and alerts to support billing compliance programs.

In some embodiments, the automated billing report generation system 300 includes a payer-specific optimization module (not illustrated) that analyzes billing code selections against the requirements and reimbursement policies of specific insurance payers. The module may recommend alternative billing code selections or additional documentation to maximize reimbursement while maintaining compliance.

In some embodiments, the automated billing report generation system 300 includes a supply and equipment tracking module (not illustrated) that identifies surgical supplies, implants, and equipment used during the procedure from the surgical video and ensures that corresponding billing codes for supplies are captured.

In some embodiments, the integrated operative report generation system 200 and the automated billing report generation system 300 include a connection layer that provides bidirectional connectivity with electronic health record (EHR) systems, practice management systems, hospital information systems, revenue cycle management systems, and claims clearinghouses. The connection layer enables automated retrieval of patient demographics, insurance information, and clinical history, and supports automated submission of claims with generated billing codes.

In some embodiments, the system 300 stores, for each billing line, a provenance record including the model version identifier for the billing code machine learning model 350, a feature extraction pipeline identifier, a rule set version identifier, a modifier determination logic version identifier, and an LLM configuration identifier. In some embodiments, the provenance record is included in the billing report 1500 and/or stored with the operative record 600 to support audits, appeals, and quality improvement. In some embodiments, the automated billing report generation system 300 includes a real-time event ledger configured to store predicted billable events as they are detected, where each event includes a timestamp range, a confidence score, one or more evidence links, and a state indicator. In some embodiments, the state indicator identifies whether the event is preliminary, verified, corrected, suppressed by rules, or flagged for review. In some embodiments, the billing report generation module 340 generates the billing report 1500 by transforming the real-time event ledger into billing lines after application of the billing rules engine and modifier determination logic.

In some embodiments, the billing report generation module 340 generates an export package that includes (i) a machine-readable representation of billing lines, (ii) a human-readable rendering, and (iii) an evidence attachment index. In some embodiments, the evidence attachment index includes links to video clips, image snapshots, and transcription excerpts stored in association with the operative record 600. In some embodiments, generating the export package reduces downstream compute by avoiding repeated media search operations and by providing a single, time-indexed evidence map.

In some embodiments, the operative report generation system 200 and the automated billing report generation system 300 include a shared indexing service configured to support low-latency retrieval of relevant media segments. In some embodiments, the shared indexing service stores embeddings for video segments and text segments and supports approximate nearest-neighbor retrieval to quickly locate supporting evidence in response to a query generated by the narrative generation module and/or the billing code verification module 330. In some embodiments, the shared indexing service supports privacy-preserving processing by storing de-identified representations of patient data and by segregating patient identifiers from media and text content. In some embodiments, patient identifiers are stored in a separate secure store with access controls, and media and transcript objects are referenced via opaque identifiers.

In some embodiments, the operative report generation system 200 and the automated billing report generation system 300 are configured to operate in a degraded connectivity mode to maintain real-time performance. In some embodiments, the degraded connectivity mode includes local buffering of audio, video, and events, and deferred upload of media and reports. In some embodiments, the operative report generation system 200 and the automated billing report generation system 300 maintain deterministic timestamping using a monotonic clock during degraded connectivity mode and later reconciles timestamps to an external time base when connectivity is restored.

In some embodiments, the operative report generation system 200 reduces surgeon interaction by automatically performing section transitions in the operative report template in response to detected phases of a procedure. In some embodiments, the detected phases are determined based on one or more of recognized spoken phrases, recognized instrument usage, recognized anatomical context, and recognized workflow patterns. In some embodiments, phase detection reduces the need for manual navigation of report sections and reduces cognitive load while maintaining chronological ordering.

In some embodiments, the system 200 reduces post-procedure editing time by generating draft text that is constrained to template fields and by maintaining links between each field and supporting evidence, thereby enabling rapid verification and targeted correction. In some embodiments, the system 200 presents a field-level confidence indicator and restricts prompts to only those fields that fall below a confidence threshold, thereby reducing interruptions and reducing total interaction events.

In some embodiments, the automated billing report generation system 300 reduces computational overhead by generating billing lines incrementally as billable events are detected rather than waiting until the end of a procedure. In some embodiments, incremental generation includes updating confidence scores and evidence links as additional corroborating information is received, and deferring only rule-set-dependent finalization steps until procedure end or until a final attestation is recorded. In some embodiments, the automated billing report generation system 300 improves technical accuracy of amount determination by coupling billing code predictions to deterministic rule evaluation and by storing a complete provenance chain for each computed amount. In some embodiments, the provenance chain links the amount to one or more of a code identifier, a modifier identifier, a rule identifier, a participant attribution record, a time interval, and one or more evidence anchors.

In some embodiments, the operative report generation system 200 and the automated billing report generation system 300 provide technical improvements including reduced end-to-end latency for operative record generation, reduced storage and compute usage for media capture through multi-stage salient selection, improved time alignment across multimodal data streams, and improved determinism and reproducibility of billing report generation through versioned rule sets and compiled rule evaluation graphs.

In some embodiments, the operative report generation system 200 and the automated billing report generation system 300 provide improved robustness in an operating room environment by reducing transcription error using speaker separation, by suppressing irrelevant audio using noise filtering, and by reducing capture error using pre-event video buffering and time alignment. In some embodiments, these improvements reduce dropped captures, mis-timestamped captures, and mis-associated evidence links.

In some embodiments, the operative report generation system 200 and the automated billing report generation system 300 provide improved computational efficiency by using streaming inference, by caching intermediate rule evaluation results, and by generating structured documents that reduce repeated parsing and transformation steps. In some embodiments, these improvements reduce processor usage and reduce memory usage during a procedure while maintaining real-time response.

In some embodiments, the operative report generation system 200 and the automated billing report generation system 300 improve technical verifiability by storing evidence anchors and provenance records for operative report text and billing report lines, thereby enabling automated and rapid consistency checking between (i) media evidence, (ii) transcriptions, (iii) operative report sections, and (iv) billing lines.

In some embodiments, the operative report generation system 200 and the automated billing report generation system 300 reduce required surgeon interaction by confining prompts to short confirmation interactions, by automatically detecting procedure phases to drive template progression, and by using confidence-driven prompting that only requests input when a confidence threshold is not satisfied, thereby enabling real-time generation with minimal interruption while preserving traceable evidence links.

In some embodiments, a system for automatic extraction of one or more salient images from a surgical video stream comprises one or more computer accessible-storage devices configured to store instructions and one or more processors communicatively connected to the one or more computer accessible storage devices and configured to execute the stored instructions to receive a plurality of records including annotated images from recorded surgical procedures to use as training data for generating an image extraction machine learning model; extract one or more first features from the training data; generate the image extraction machine learning model, by training the image extraction machine learning model on the extracted one or more first features, to output salient images in the training data; receive the surgical video stream; extract one or more second features from the surgical video stream; and input the one or more second features into the trained image extraction machine learning model to output the one or more salient images from the surgical video stream.

In some embodiments, the system is further configured to automatically generate a surgical operative record including at least the extracted one or more salient images from the surgical video stream.

In some embodiments, the system is further configured to receive a plurality of annotated speech samples to use as training data for generating a speech transcription machine learning model; extract one or more third features from the training data; generate the speech machine learning model, by training the speech machine learning model on the extracted one or more third features, to output a speech transcription; receive an audio stream associated with the surgical video stream; extract one or more fourth features from the audio stream; input the one or more fourth features into the trained speech machine learning model to output a speech transcription of the audio stream; and associate the extracted one or more salient images with the output speech transcription.

In some embodiments, the system is further configured to automatically generate a surgical operative record including at least the one or more extracted salient images and the associated output speech transcription.

In some embodiments, a timestamp is associated with each salient image of the one or more salient images.

In some embodiments, the system is further configured to receive a plurality of records including annotated video clips from recorded surgical procedures to use as second training data for training the image extraction machine learning model; extract one or more fifth features from the second training data; train the image extraction machine learning model, using the extracted one or more fifth features, to output salient video clips in the second training data; extract one or more sixth features from the surgical video stream; and input the one or more sixth features into the trained image extraction machine learning model to output one or more salient video clips from the surgical video stream.

In some embodiments, a processor implemented method of automatically extracting salient images from a surgical video stream comprises receiving a plurality of records including annotated images from recorded surgical procedures to use as training data for generating an image extraction machine learning model; extracting one or more first features from the training data; generating the image extraction machine learning model, by training the image extraction machine learning model on the extracted one or more first features, to output salient images in the training data; receiving the surgical video stream; extracting one or more second features from the surgical video stream; and inputting the one or more second features into the trained image extraction machine learning model to output the one or more salient images from the surgical video stream.

In some embodiments, the method further comprises automatically generating a surgical operative record including at least the extracted one or more salient images from the surgical video stream.

In some embodiments, the method further comprises receiving a plurality of annotated speech samples to use as training data for generating a speech transcription machine learning model; extracting one or more third features from the training data; generating the speech machine learning model, by training the speech machine learning model on the extracted one or more third features, to output a speech transcription; receiving an audio stream associated with the surgical video stream; extracting one or more fourth features from the audio stream; inputting the one or more fourth features into the trained speech machine learning model to output a speech transcription of the audio stream; and associating the extracted one or more salient images with the output speech transcription.

In some embodiments, the method further comprises automatically generating a surgical operative record including at least the one or more extracted salient images and the associated output speech transcription.

In some embodiments, the method further comprises associating a timestamp with each salient image of the one or more salient images.

In some embodiments, the method further comprises receiving a plurality of records including annotated video clips from recorded surgical procedures to use as second training data for generating the image extraction machine learning model; extracting one or more fifth features from the second training data; training the image extraction machine learning model, using the extracted one or more fifth features, to output salient video clips in the second training data; extracting one or more sixth features from the surgical video stream; and inputting the one or more sixth features into the trained image extraction machine learning model to output one or more salient video clips in the surgical video stream.

In some embodiments, an operative record generation system comprises one or more computer accessible-storage devices configured to store instructions and one or more processors communicatively connected to the one or more computer accessible storage devices and configured to execute the stored instructions to provide a plurality of program modules including a user interface, a speech processing module, an image processing module, and a record generation module.

The user interface is configured to display a video of the surgical procedure in a first portion of a screen and a transcription of a user's speech in a second portion of the screen. The speech processing module is configured to identify one or more predetermined keywords in the transcription, each keyword associated with a particular function executed by the operative record generation system. The image processing module is configured to, in a case where the speech processing module identifies a keyword associated with an image capture or video capture function, record an image or a portion of the video of the operation displayed on the screen and a timestamp associated with the recorded image or the portion of the video. The record generation module is configured to generate the operative record using the transcription of the user's speech and the recorded image or the portion of the video.

In some embodiments, the user interface further includes setting screens configured to personalize settings of the speech processing module for a user.

In some embodiments, the user interface is further configured to display an indicator indicating an active or inactive status of the operative record generation system.

In some embodiments, the speech processing module is further configured to extract the user's speech by filtering an input audio stream to remove background noise and non-user speech.

In some embodiments, the video and image processing module is further configured to receive a plurality of operative records including tagged videos and images to use as training data for generating a video and image extraction machine learning model; extract one or more features from the training data; and train the video and image extraction machine learning model, using the extracted one or more features, to automatically record an image or a portion of the video of the operation displayed on the screen and a timestamp associated with the recorded image or the portion of the video.

In some embodiments, the system further includes a speech transcription module configured to receive a plurality of transcribed and annotated speech samples to use as training data for generating a speech transcription machine learning model; extract one or more features from the training data; and train the speech machine learning model, using the extracted one or more features, to transcribe the user's speech.

In some embodiments, a method of generating an operative record includes displaying, on a user interface, a video of the surgical procedure in a first portion of a screen and a transcription of a user's speech in a second portion of the screen; identifying one or more predetermined keywords in the transcription, each keyword associated with a particular function executed by the operative record generation system; in a case where a keyword associated with an image capture or video capture function is identified, recording an image or a portion of the video of the operation displayed on the screen and a timestamp associated with the recorded image or the portion of the video; and generating the operative record using the transcription of the user's speech and the recorded image or the portion of the video.

In some embodiments, the method further includes extracting the user's speech by filtering an input audio stream to remove background noise and non-user speech.

In some embodiments, the method further includes receiving a plurality of operative records including tagged videos and images to use as training data for generating a video and image extraction machine learning model; extracting one or more features from the training data; and training the video and image extraction machine learning model, using the extracted one or more features, to automatically record an image or a portion of the video of the operation displayed on the screen and a timestamp associated with the recorded image or the portion of the video.

In some embodiments, the method further includes receiving a plurality of transcribed and annotated speech samples to use as training data for generating a speech transcription machine learning model; extracting one or more features from the training data; and training the speech machine learning model, using the extracted one or more features, to transcribe the user's speech.

In some embodiments, a system for generating a billing report for a surgical procedure, comprises one or more computer accessible storage devices configured to store instructions, and one or more processors communicatively connected to the one or more computer accessible storage devices and configured to execute the instructions to receive at least one of a surgical video stream or a surgical audio stream for the surgical procedure; generate, when the surgical audio stream is received, a transcription stream from the surgical audio stream using incremental decoding that produces partial transcriptions and updates the partial transcriptions as additional audio samples are received; extract a plurality of multimodal features from at least one of the surgical video stream, the surgical audio stream, or the transcription stream; input the plurality of multimodal features into a trained billing code machine learning model to output a set of predicted billing codes each having associated therewith at least one of (i) a timestamp or time range in the surgical video stream or (ii) a confidence score; determine, based on a case context derived from at least one of scheduling metadata, encounter metadata, the transcription stream, or the surgical video stream, one or more modifiers applicable to at least one predicted billing code; select a versioned rule set for at least a subset of the predicted billing codes, the versioned rule set defining one or more of (i) eligibility conditions, (ii) modifier effects, (iii) bundling or unbundling outcomes, or (iv) amount computation rules; transform the set of predicted billing codes into a plurality of billing lines by at least applying the versioned rule set and the one or more modifiers to the predicted billing codes; and generate the billing report including the plurality of billing lines.

In some embodiments, when the system for generating the billing report receives both a surgical video stream and a surgical audio stream for a surgical procedure, the incremental decoding used to generate the transcription stream produces partial transcriptions that are time-aligned to the surgical video stream. In some embodiments, each token, word, or phrase in the transcription stream is associated with a timestamp referenced to a common time base used by the surgical video stream such that the transcription stream can be mapped deterministically to individual frames or frame ranges of the surgical video stream.

In some embodiments, the incremental decoding for the transcription stream is implemented using a streaming inference pipeline that provides bounded-latency updates. In some embodiments, the streaming inference pipeline maintains a bounded-latency buffer of audio samples and generates partial transcriptions within a predetermined latency budget while updating the partial transcriptions as additional audio samples are received. In some embodiments, the combination of the bounded-latency streaming inference pipeline and the time-aligned timestamps enables the billing report generation system to associate predicted billing codes and billing lines with precise temporal locations in the surgical video stream and the transcription stream in real time or near real time.

In some embodiments, the one or more processors are further configured to execute the instructions to compile the versioned rule set into an evaluation graph; and execute the evaluation graph using cached intermediate results to transform the set of predicted billing codes into the plurality of billing lines, wherein execution of the evaluation graph includes at least one of (i) applying one or more eligibility conditions or one or more bundling or unbundling rules to suppress one or more of the predicted billing codes, add one or more new billing codes to the predicted billing codes, or replace a subset of the predicted billing codes with a different set of billing codes, (ii) applying one or more modifier effects to adjust at least one amount computation rule for generating a billing amount associated with a billing line of the plurality of billing lines, or (iii) computing, for each billing line of the plurality of billing lines, the billing amount or an allocation of the billing amount among a plurality of participants in the surgical procedure.

In some embodiments, the plurality of participants include two or more of a primary surgeon, an assistant surgeon, a co-surgeon, a resident, a fellow, an anesthesiologist, a certified registered nurse anesthetist, a circulating nurse, a scrub nurse, or a technician, and generating the plurality of billing lines comprises generating a participant attribution record for at least one billing line.

In some embodiments, the one or more processors are further configured to execute the instructions to determine participant roles using at least one of scheduled staffing data, electronic health record participation logs, badge-in and badge-out logs, intraoperative device usage logs, transcribed speech indicating role participation, or video-based recognition of role indicators.

In some embodiments, the cached intermediate results include at least one cached condition evaluation that is reused for generating a plurality of billing lines to reduce end-to-end billing report generation latency.

In some embodiments, determining the one or more modifiers comprises determining an emergent-versus-scheduled modifier in response to detecting at least one emergent workflow indicator in at least one of the scheduling metadata, the encounter metadata, the transcription stream, or the surgical video stream.

In some embodiments, determining the one or more modifiers comprises resolving conflicts among a plurality of candidate modifiers using a stored modifier precedence table associated with at least one of a payer identifier or an institution identifier.

In some embodiments, the one or more processors are further configured to execute the instructions to store, for at least one billing line of the plurality of billing lines, a provenance record identifying at least one of a model version identifier for the trained billing code machine learning model, a feature extraction pipeline identifier, the versioned rule set, or a modifier determination logic version identifier.

In some embodiments, the billing report includes a facility billing portion and a professional billing portion, each portion including one or more of the plurality of billing lines.

In some embodiments, a system for deterministic rule-based computation of billing lines for a surgical procedure comprises one or more processors and one or more computer accessible storage devices storing instructions that, when executed by the one or more processors, cause the system to receive a set of candidate billing codes for the surgical procedure, each candidate billing code being associated with at least one evidence item comprising a timestamp range, a video segment identifier, an audio segment identifier, or a transcription excerpt identifier; select a versioned billing rule set based on at least one of an institution identifier, a payer identifier, a specialty identifier, or a site-of-service identifier; compile the versioned billing rule set into an evaluation graph having (i) nodes representing rule conditions or rule outcomes and (ii) edges representing dependencies between the rule conditions and the rule outcomes; execute the evaluation graph to compute, for each billing line of the plurality of billing lines, an amount or an allocation of an amount among a plurality of participants; and output the plurality of billing lines with retained associations to the evidence items.

In some embodiments, compiling the versioned billing rule set comprises generating the evaluation graph in a representation that enables deterministic traversal order to improve reproducibility of amount computation across repeated executions.

In some embodiments, executing the evaluation graph comprises caching at least one intermediate result and reusing the cached intermediate result for computing amounts for multiple billing lines of the plurality of billing lines.

In some embodiments, the instructions further cause the system to generate, for at least one billing line, a compliance flag in response to a determination that an evidence requirement associated with the versioned billing rule set is not satisfied.

In some embodiments, the instructions further cause the system to store an identifier of the versioned billing rule set in association with the output billing lines to provide an audit trail.

In some embodiments, a system for generating an evidence index for billing report generation for a surgical procedure comprises one or more processors and one or more computer accessible storage devices storing instructions that, when executed by the one or more processors, cause the system to synchronize a surgical video stream, an associated audio stream, and a transcription stream into a unified procedure timeline having a common time base; generate an index mapping time ranges to corresponding video segments, audio segments, transcription excerpts, predicted billing codes, and billing lines; and use the index to populate evidence links in a billing report by retrieving, in response to a billing line identifier, at least one linked video segment, audio segment, or transcription excerpt without re-searching the surgical video stream.

In some embodiments, the synchronizing comprises compensating for buffering delay by estimating at least one offset parameter using a calibration signal detectable in at least one of the associated audio stream or the surgical video stream.

In some embodiments, the index stores opaque identifiers that reference media objects stored separately from patient identifiers to support privacy-preserving processing.

In some embodiments, the instructions further cause the system to store, in association with the billing report, an evidence attachment index that maps each billing line to a corresponding set of media identifiers and time ranges.

In some embodiments, a system for incremental generation of billing report lines during a surgical procedure comprises one or more processors and one or more computer accessible storage devices storing instructions that, when executed by the one or more processors, cause the system to maintain a real-time event ledger that stores predicted billable events as the predicted billable events are detected from at least one of a surgical video stream, an associated audio stream, or a transcription stream, each predicted billable event including a timestamp range, a confidence score, at least one evidence link, and a state indicator; update a predicted billable event from a preliminary state to a verified state in response to receiving corroborating evidence; apply a versioned billing rule set to the real-time event ledger to generate a plurality of billing lines including computed amounts or allocations; and generate a billing report by transforming entries of the real-time event ledger into the billing lines after application of the versioned billing rule set.

In some embodiments, applying the versioned billing rule set comprises suppressing at least one ledger entry by changing the state indicator to a suppressed-by-rules state while retaining the at least one evidence link for auditability.

In some embodiments, the instructions further cause the system to defer at least one rule-set-dependent finalization step until receipt of a procedure completion indicator while continuing to update evidence links during the surgical procedure.

In some embodiments, the instructions further cause the system to store a reconciliation record identifying at least one difference between (i) preliminary billing lines generated during the surgical procedure and (ii) final billing lines generated after receipt of additional evidence.

In some embodiments, a system for generating evidence-anchored billing justifications for a surgical procedure comprises one or more processors and one or more computer accessible storage devices storing instructions that, when executed by the one or more processors, cause the system to receive a plurality of billing lines, each billing line including a billing code and at least one associated evidence item comprising a timestamp range, a video segment identifier, an audio segment identifier, or a transcription excerpt identifier; select a versioned phrase library based on at least one of a payer identifier, an institution identifier, or a specialty identifier; generate, using a large language model, a justification text field for at least one billing line by conditioning generation on (i) the billing code, (ii) a subset of evidence items associated with the billing line, and (iii) one or more standardized phrases retrieved from the versioned phrase library; and enforce an evidence-anchoring constraint in which each sentence of the justification text field includes at least one reference to at least one of the evidence items, and, in response to determining that the evidence-anchoring constraint is not satisfied, regenerate the justification text field or mark the billing line for human review.

In some embodiments, the standardized phrases include at least one rule-required phrase that is inserted in response to a determination that a rule condition in a versioned billing rule set is satisfied for the billing line.

In some embodiments, enforcing the evidence-anchoring constraint comprises rejecting at least one candidate justification sentence that lacks a reference to an evidence item and replacing the rejected candidate justification sentence with a different sentence that includes a reference to an evidence item.

In some embodiments, the instructions further cause the system to store an identifier of the versioned phrase library in association with the billing report to provide an audit trail.

In some embodiments, a system for evidence sufficiency evaluation in billing report generation for a surgical procedure comprises one or more processors and one or more computer accessible storage devices storing instructions that, when executed by the one or more processors, cause the system to receive a plurality of billing lines each associated with at least one evidence link; determine, using an evidence sufficiency analyzer, an evidence sufficiency status for at least one billing line based on an evidence requirement of a versioned billing rule set; and in response to determining that evidence is insufficient, mark the billing line as requiring review and store a description of at least one missing evidence item.

In some embodiments, the evidence requirement includes a minimum-duration requirement for a linked video segment and wherein determining the evidence sufficiency status comprises computing a duration from timestamps associated with the linked video segment.

In some embodiments, the evidence requirement includes presence of a required phrase in a transcription excerpt and wherein determining the evidence sufficiency status comprises identifying the required phrase in the transcription excerpt.

In some embodiments, the instructions further cause the system to generate an alert comprising a recommended corrective action that identifies at least one additional evidence item to capture or to link.

In some embodiments, a system for evaluating billing rule updates for billing report generation comprises one or more processors and one or more computer accessible storage devices storing instructions that, when executed by the one or more processors, cause the system to store a frozen evidence snapshot for a surgical procedure, the frozen evidence snapshot comprising (i) a set of candidate billing codes and (ii) evidence items associated with the candidate billing codes; apply, using a billing rules engine, a first versioned rule set to the frozen evidence snapshot to generate a first set of billing lines including computed amounts or allocations; apply, using the billing rules engine, a second versioned rule set different from the first versioned rule set to the frozen evidence snapshot to generate a second set of billing lines including computed amounts or allocations; compare the first set of billing lines to the second set of billing lines to determine at least one difference comprising a difference in a billing code, a modifier, an amount, an allocation, or an evidence requirement; and generate an output report identifying the at least one difference and including at least one evidence link corresponding to the at least one difference.

In some embodiments, the frozen evidence snapshot is stored with an identifier of a billing code machine learning model version and an identifier of a feature extraction pipeline to enable repeatable evaluation.

In some embodiments, the comparing comprises determining that a difference is attributable to a rule change by confirming that the frozen evidence snapshot is identical for the first versioned rule set and the second versioned rule set.

In some embodiments, generating the output report comprises generating a machine-readable representation that identifies the first versioned rule set and the second versioned rule set.

In some embodiments, a system for low-latency evidence retrieval for billing report generation comprises one or more processors and one or more computer accessible storage devices storing instructions that, when executed by the one or more processors, cause the system to store embeddings for video segments and text segments derived from a surgical video stream and an associated transcription stream; receive a query derived from a billing line; retrieve, using approximate nearest-neighbor search over the embeddings, candidate evidence items for the billing line; and populate at least one evidence link in a billing report using at least one of the candidate evidence items.

In some embodiments, the query is derived from at least one of a billing code, a modifier, or a rule outcome associated with the billing line.

In some embodiments, the instructions further cause the system to store de-identified embeddings separately from patient identifiers and to reference media objects using opaque identifiers.

In some embodiments, a system for generating an export package for a billing report for a surgical procedure comprises one or more processors and one or more computer accessible storage devices storing instructions that, when executed by the one or more processors, cause the system to generate a billing report comprising a plurality of billing lines; generate an evidence attachment index mapping the plurality of billing lines to corresponding evidence links; generate a machine-readable representation of the plurality of billing lines and a human-readable rendering of the billing report; and output an export package that includes the machine-readable representation, the human-readable rendering, and the evidence attachment index.

In some embodiments, the machine-readable representation conforms to at least one interoperability mapping associated with a destination system identifier.

In some embodiments, generating the export package comprises generating a time-indexed evidence map configured to reduce repeated media search operations by downstream systems.

In some embodiments, a processor-implemented method of generating a billing report for a surgical procedure comprises receiving at least one of a surgical video stream or a surgical audio stream for the surgical procedure; generating, when the surgical audio stream is received, a transcription stream from the surgical audio stream using incremental decoding that produces partial transcriptions and updates the partial transcriptions as additional audio samples are received; extracting a plurality of multimodal features from at least one of the surgical video stream, the surgical audio stream, or the transcription stream; inputting the plurality of multimodal features into a trained billing code machine learning model to output a set of predicted billing codes each having associated therewith at least one of (i) a timestamp or time range in the surgical video stream or (ii) a confidence score; determining, based on a case context derived from at least one of scheduling metadata, encounter metadata, the transcription stream, or the surgical video stream, one or more modifiers applicable to at least one predicted billing code; selecting a versioned rule set for at least a subset of the predicted billing codes, the versioned rule set defining one or more of (i) eligibility conditions, (ii) modifier effects, (iii) bundling or unbundling outcomes, or (iv) amount computation rules; transforming the set of predicted billing codes into a plurality of billing lines by at least applying the versioned rule set and the one or more modifiers to the predicted billing codes; and generating the billing report including the billing lines.

In some embodiments, a processor-implemented method of generating an evidence-anchored justification for a billing line in a billing report for a surgical procedure comprises receiving a billing line including a billing code and an evidence item comprising a timestamp range, a video segment identifier, an audio segment identifier, or a transcription excerpt identifier; selecting a versioned phrase library; generating, using a large language model, a justification text field for the billing line conditioned on the billing code, the evidence item, and at least one standardized phrase from the versioned phrase library; verifying that each sentence of the justification text field includes a reference to an evidence item; and in response to determining that a sentence lacks a reference to an evidence item, regenerating the justification text field or marking the billing line for review.

In some embodiments, a system for verification of a billing report for a surgical procedure comprises one or more processors and one or more computer accessible storage devices storing instructions that, when executed by the one or more processors, cause the system to receive a surgical video stream and an associated audio stream; receive a set of proposed billing lines; generate, using a trained billing code machine learning model and a billing rules engine applying a versioned rule set, a set of predicted billing lines for the surgical procedure; compare the set of predicted billing lines to the set of proposed billing lines to determine at least one discrepancy; and generate a verification report identifying the at least one discrepancy and including at least one evidence link supporting the discrepancy.

In some embodiments, the discrepancy includes at least one of a missing billing code, an extra billing code, an incorrect modifier, an incorrect participant allocation, an incorrect computed amount, or a missing evidence item.

In some embodiments, one or more computer non-transitory storage media are configured to store one or more programs that include instructions for executing one or more of the various methods executed by the systems discussed above.

Subsets or combinations of various embodiments described above provide further embodiments. These and other changes can be made to the invention in light of the above-detailed description and still fall within the scope of the present invention. In general, in the following claims, the terms used should not be construed to limit the invention to the specific embodiments disclosed in the specification. Accordingly, the invention is not limited by the disclosure, but instead its scope is to be determined entirely by the following claims.

1. A system for generating a billing report for a surgical procedure, comprising:
one or more computer accessible storage devices configured to store instructions; and
one or more processors communicatively connected to the one or more computer accessible storage devices and configured to execute the instructions to:
receive at least one of a surgical video stream or a surgical audio stream for the surgical procedure;
generate, when the surgical audio stream is received, a transcription stream from the surgical audio stream using incremental decoding;
extract a plurality of multimodal features from at least one of the surgical video stream, the surgical audio stream, or the transcription stream;
input the plurality of multimodal features into a trained billing code machine learning model to output a set of predicted billing codes each having associated therewith at least one of (i) a timestamp or time range or (ii) a confidence score;
determine, based on a case context derived from at least one of scheduling metadata, encounter metadata, the transcription stream, or the surgical video stream, one or more modifiers applicable to at least one predicted billing code;
select a versioned rule set for at least a subset of the predicted billing codes, the versioned rule set defining one or more of (i) eligibility conditions, (ii) modifier effects, (iii) bundling or unbundling outcomes, or (iv) amount computation rules;
transform the set of predicted billing codes into a plurality of billing lines by at least applying the versioned rule set and the one or more modifiers to the predicted billing codes; and
generate the billing report including the plurality of billing lines.

2. The system of claim 1,
wherein receiving the at least one of the surgical video stream or the surgical audio stream comprises receiving both the surgical video stream and the surgical audio stream, and
wherein generating the transcription stream comprises time-aligning the transcription stream to the surgical video stream.

3. The system of claim 1, wherein generating the transcription stream using incremental decoding comprises producing partial transcriptions at a bounded latency and revising at least one partial transcription in response to receipt of additional audio samples.

4. The system of claim 1, wherein extracting the plurality of multimodal features comprises extracting at least one video feature comprising at least one of instrument features, anatomical structure features, motion features, or action recognition features.

5. The system of claim 1, wherein extracting the plurality of multimodal features comprises extracting at least one audio-derived feature comprising at least one of a keyword feature, a medical entity feature, a semantic embedding feature, or a temporal alignment feature between a transcription excerpt and a time range.

6. The system of claim 1, wherein the trained billing code machine learning model comprises a multimodal neural network configured to fuse a video feature representation and a transcription feature representation to output the set of predicted billing codes.

7. The system of claim 1, wherein determining the one or more modifiers comprises determining an emergent-versus-scheduled modifier in response to detecting at least one emergent workflow indicator in at least one of the scheduling metadata, the encounter metadata, the transcription stream, or the surgical video stream.

8. The system of claim 1, wherein determining the one or more modifiers comprises resolving conflicts among a plurality of candidate modifiers using a stored modifier precedence table associated with at least one of a payer identifier or an institution identifier.

9. The system of claim 1, wherein selecting the versioned rule set comprises selecting the versioned rule set based on at least one of an institution identifier, a payer identifier, a specialty identifier, or a site-of-service identifier.

10. The system of claim 1, wherein transforming the set of predicted billing codes into the plurality of billing lines comprises applying at least one eligibility condition of the versioned rule set to suppress at least one predicted billing code.

11. The system of claim 1, wherein transforming the set of predicted billing codes into the plurality of billing lines comprises applying at least one bundling or unbundling outcome of the versioned rule set to replace at least one predicted billing code with a different billing code or to add at least one additional billing code.

12. The system of claim 1, wherein the amount computation rules comprise participant allocation rules that allocate at least a portion of an amount among a plurality of participants of the surgical procedure.

13. The system of claim 12, wherein the plurality of participants includes two or more of a primary surgeon, an assistant surgeon, a co-surgeon, a resident, a fellow, an anesthesiologist, a certified registered nurse anesthetist, a circulating nurse, a scrub nurse, or a technician, and wherein generating the billing report comprises generating a participant attribution record identifying a participant for at least one billing line.

14. The system of claim 13, wherein the one or more processors are further configured to determine participant roles using at least one of scheduled staffing data, electronic health record participation logs, badge-in and badge-out logs, intraoperative device usage logs, transcribed speech indicating role participation, or video-based recognition of role indicators.

15. The system of claim 1,
wherein the versioned rule set is compiled into an evaluation graph configured for deterministic traversal order, and
wherein transforming the set of predicted billing codes into the plurality of billing lines comprises executing the evaluation graph using cached intermediate results reused across a plurality of billing lines to reduce end-to-end billing report generation latency.

16. The system of claim 1, wherein the billing report includes, for each billing line, at least one of a timestamp or time range, a confidence score, or an evidence link that references at least one of a video segment identifier, an audio segment identifier, or a transcription excerpt identifier.

17. The system of claim 1, wherein the one or more processors are further configured to store, in association with at least one billing line, a provenance record identifying at least one of a model version identifier for the trained billing code machine learning model, a feature extraction pipeline identifier, a version identifier of the versioned rule set, or a modifier determination logic version identifier.

18. The system of claim 1,
wherein the one or more processors are further configured to generate, using a large language model, a justification text field for at least one billing line, and
wherein generating the justification text field comprises inserting at least one standardized phrase from a versioned phrase library and enforcing an evidence-anchoring constraint in which each sentence of the justification text field includes a reference to at least one evidence item.

19. A processor-implemented method of generating a billing report for a surgical procedure, comprising:
receiving at least one of a surgical video stream or a surgical audio stream for the surgical procedure;
generating, when the surgical audio stream is received, a transcription stream from the surgical audio stream using incremental decoding;
extracting a plurality of multimodal features from at least one of the surgical video stream, the surgical audio stream, or the transcription stream;
inputting the plurality of multimodal features into a trained billing code machine learning model to output a set of predicted billing codes each having associated therewith at least one of (i) a timestamp or time range or (ii) a confidence score;
determining, based on a case context derived from at least one of scheduling metadata, encounter metadata, the transcription stream, or the surgical video stream, one or more modifiers applicable to at least one predicted billing code;
selecting a versioned rule set for at least a subset of the predicted billing codes, the versioned rule set defining one or more of (i) eligibility conditions, (ii) modifier effects, (iii) bundling or unbundling outcomes, or (iv) amount computation rules;
transforming the set of predicted billing codes into a plurality of billing lines by at least applying the versioned rule set and the one or more modifiers to the predicted billing codes; and
generating the billing report including the plurality of billing lines.

20. A non-transitory computer-readable storage medium storing instructions that, when executed by one or more processors, cause the one or more processors to perform a method of generating a billing report for a surgical procedure, the method comprising:
receiving at least one of a surgical video stream or a surgical audio stream for the surgical procedure;
generating, when the surgical audio stream is received, a transcription stream from the surgical audio stream using incremental decoding;
extracting a plurality of multimodal features from at least one of the surgical video stream, the surgical audio stream, or the transcription stream;
inputting the plurality of multimodal features into a trained billing code machine learning model to output a set of predicted billing codes each having associated therewith at least one of (i) a timestamp or time range or (ii) a confidence score;
determining, based on a case context derived from at least one of scheduling metadata, encounter metadata, the transcription stream, or the surgical video stream, one or more modifiers applicable to at least one predicted billing code;
selecting a versioned rule set for at least a subset of the predicted billing codes, the versioned rule set defining one or more of (i) eligibility conditions, (ii) modifier effects, (iii) bundling or unbundling outcomes, or (iv) amount computation rules;
transforming the set of predicted billing codes into a plurality of billing lines by at least applying the versioned rule set and the one or more modifiers to the predicted billing codes; and
generating the billing report including the plurality of billing lines.
